(19) United States
(12) Patent Application Publication
(10) Pub. No.: US 2025/0213748 A1
(43) Pub. Date: Jul. 3, 2025

(54) DEVICES AND METHODS FOR STORING AND APPLYING BONE GROWTH COMPOSITIONS

(71) Applicant: Warsaw Orthopedic, Inc., Warsaw, IN (US)

(72) Inventors: Benjamin T. Reves, Memphis, TN (US); Daniel A. Shimko, Germantown, TN (US); David R. Kaes, Toms River, NJ (US)

(21) Appl. No.: 18/986,901

(22) Filed: Dec. 19, 2024

Related U.S. Application Data
(60) Provisional application No. 63/615,896, filed on Dec. 29, 2023.

(51) Int. Cl. A61L 27/36; A61F 2/46; A61L 27/54

(52) U.S. Cl. CPC A61L 27/3608; A61F 2/4601; A61L 27/365; A61L 27/54; A61F 2310/00359; A61L 2300/412; A61L 2400/06; A61L 2430/02

(57) ABSTRACT

Methods and devices for applying and handling a bone growth composition are provided. In certain embodiments, an application device may be provided that may be loaded or filled with thawed bone growth composition or mixtures including the thawed bone growth composition. The application device may be used in dispensing or localizing the bone growth composition at an anatomic site of interest.

### CROSS-REFERENCE TO RELATED APPLICATION

This application claims priority and benefit of U.S. Provisional Patent Application No. 63/615,896, entitled “DEVICES AND METHODS FOR STORING AND APPLYING BONE GROWTH COMPOSITIONS,” filed on Dec. 29, 2023, which is incorporated herein by reference in its entirety for all purposes.

### TECHNICAL FIELD

The present disclosure relates generally to bone growth compositions, such as cellular bone matrix compositions, and devices and methodologies associated with storing, transporting, and/or dispensing or applying such bone growth compositions.

### BACKGROUND

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In clinical use, a variety of conditions may warrant repair and/or replacement of an internal body part, such as bone. For example, to repair a bone fracture, an adhesive agent may be applied to adhere sections of the separated bone together. A bone filler material may be applied to a bone in a subject to replace degenerated tissue and/or to provide a supportive matrix to support or reinforce the bone and promote bone growth.

Since the early 1990s, carefully processed allograft bone from donated human tissues have been routinely transformed into various type of bone grafting materials with many derivations in shape, form, and purpose. The first standards for tissue banking published by the American Association of Tissue Banks (AATB) in 1984. Due to an increasing medical and market demand for orthopedic implants, a selection of grafting options has been developed. Fresh frozen allograft bone (derived from either deceased donors or surgical discards) has been used to supplement the available autograft bone recovered from patients undergoing surgery and in need of bone grafting. Osteotech launched the first formulated demineralized bone matrix product for bone grafting called Grafton™ Gel in 1991.

Commercial products intended as an alternative to autologous grafts have included growth factors, or a combination of natural or synthetic scaffold materials. While the use of demineralized bone fibers (DBF) creates structural matrices and promotes bone growth, the use of DBF alone lacks the viability of fresh tissue. Certain techniques may use cell-containing bone materials. However, clinical use of cell bone matrices has been associated with significant issues, such as high cost, burdensome and nonoverlapping regulatory matters globally, storage and transport challenges, and may infer complications associated with immunological response.

Cellular matrix products manufactured in the United States are regulated by section 361 of the Public Health Service Act and Code of Federal Regulation (CFR) title 21 section 1271, which does not require Food and Drug Administration (FDA) premarket review and approval. As such, the products will be considered human cells, tissues, or cellular or tissue-based products (HCT/Ps) so long as the products satisfy the following criteria: minimal manipulation; homologous use only; systemic effect absence; the primary function not dependent on the metabolic activity of viable cells, unless the product is intended for autologous use or use by a first- or second-degree blood relative.

For each CBM, however, several intrinsic biological characteristics, such as viable cell sources, the donor age at the time of graft harvest, or cell survival after transplantation, cause variations among different lots of the same product in terms of expected outcomes. Processing may also introduce variability in product; such variability may include cell type, cell amount, cell viability dependent on donor material, cell viability after thawing, bone tissue processing, subsequent formulation, cryoprotectant agents, cryopreservation or freezing, and storage.

Further issues in regards to maintaining viability of cells remain. Cryoprotectant agents utilized to maintain cell viability at extremely low temperatures (e.g., less than about −80° C.) and for long-term storage and transport, e.g., dimethyl sulfoxide (DMSO), have an intrinsic cytotoxicity that requires rapid removal from grafts before implant. In fact, cryoprotectants during thaw cause cell death, therefore requiring additional rinsing, decanting, and handling that may be detrimental to the viable cells. Use of other cryoagents, or storing at less extreme cold temperatures has caused shrinking of the cells and/or caused formation of intracellular ice (which causes the cell to burst upon thaw).

A need exists to develop a CBM/VBM product that maintains a cellular viability similar to the live cell count of the extracted bone particles, granules, or fibers prior to processing. A CBM product is needed that will address the issues as described above.

### SUMMARY

Certain embodiments are summarized below. These embodiments are not intended to limit the scope of the disclosure. Indeed, the present disclosure may encompass a variety of forms that may be similar to or different from the embodiments set forth below. The bone growth composition of the current application will address the issues discussed prior, and further include an improved capability for use, manipulation, preservation, thaw and post-thaw applications. Such improvements are described herein.

In one embodiment, a package is provided. In accordance with this embodiment, the package comprises an inner pouch comprising a bone growth composition within a sealed and sterile environment. The bone growth composition comprises: demineralized bone fibers generated from a donor bone tissue; cancellous chips and/or corticocancellous chips generated from the donor bone tissue; and a cryosolution (e.g., a cryopreservative) comprising glycerol in Lactated Ringer's solution, wherein a glycerol content of the cryosolution is equal to or less than 25% by volume.

In accordance with aspects of the above embodiment, the package further comprises a pouch within which the inner pouch is sealed or placed. In accordance with certain such embodiments, the package further comprises an outer pouch within which the pouch is sealed or placed. In accordance with certain additional embodiments the pouch is configured to be opened by one of having two layers forming the pouch being peeled apart, being cut open, or being torn open along a perforation provided on the pouch. In accordance with certain additional embodiments the pouch further comprises a rubberized seal configured to allow insertion of a temperature modulating medium internal to the pouch but external to the inner pouch. In accordance with certain additional embodiments the inner pouch comprises one or more drainage features that, when exposed, facilitate drainage of the cryosolution from the inner pouch. In accordance with certain additional embodiments the inner pouch and pouch are formed integral with one another. In accordance with certain additional embodiments the inner pouch further comprises a resealing feature configured to allow resealing of the inner pouch after opening. In accordance with certain further embodiments the inner pouch is configured to be opened by one of having two layers forming the inner pouch being peeled apart, being cut open, or being torn open along a perforation provided on the inner pouch. In accordance with certain further embodiments the inner pouch contains 1 cc, 2.5 cc, 5 cc, 10, cc, or 15 cc of bone growth composition. In accordance with certain further embodiments, the package further comprises: a sealed cannula tray; and a die card provided within the cannula tray, wherein a plurality of cannulas is held within the die card and wherein each cannula is configured to receive the bone growth composition or a combination of the bone growth composition and one or both of local autograft or bone marrow aspirate.

In one embodiment a method of loading a cannula is provided. In accordance with this embodiment, an amount of bone growth composition is prepared for loading into an application device. The amount of bone growth composition or a combination of the bone growth composition and one or both of local autograft or bone marrow aspirate is inserted into the cannula. The bone growth composition comprises: demineralized bone fibers generated from a donor bone tissue; cancellous chips and/or corticocancellous chips generated from the donor bone tissue; and a cryosolution comprising glycerol in Lactated Ringer's solution, wherein a glycerol content of the cryosolution is equal to or less than 25% by volume.

In accordance with aspects of the above embodiment, preparing the amount of bone growth composition comprises mixing the bone growth composition and one or both of local autograft or bone marrow aspirate prior to insertion into the cannula. In accordance with certain additional embodiments the method further comprises, prior to inserting the amount of bone growth composition or the combination of bone growth composition and one or both of local autograft or bone marrow aspirate into the cannula, positioning the cannula on a loading tray, wherein the loading tray comprises at least one groove configured to stabilize the cannula when positioned on the loading tray. In accordance with certain additional embodiments the method further comprises closing the cannula once filled with the bone growth composition or the combination of bone growth composition and one or both of local autograft or bone marrow. In accordance with certain additional embodiments the method further comprises, after insertion of the bone growth composition or the combination of bone growth composition and one or both of local autograft or bone marrow, inserting the cannula into a delivery tube of an application device; securing the delivery tube containing the cannula to the application device; and operating the application device to dispense bone growth composition or the combination of bone growth composition and one or both of local autograft or bone marrow from the cannula to an anatomic site of a patient. In accordance with certain additional embodiments inserting the amount of bone growth composition or the combination of the bone growth composition and one or both of local autograft or bone marrow aspirate into the cannula comprises autoloading the cannula with a measured amount of bone growth composition and one or both of local autograft or bone marrow aspirate according to volumetric measurement marks provided on the cannula. In accordance with certain additional embodiments the application device is a syringe or a bone-grafting injection gun. In accordance with certain additional embodiments the anatomic site comprises a spine region or the anatomic site comprises an area of the patient anatomy comprising a bone defect to be repaired.

In one embodiment a method of applying a bone growth composition is provided. In accordance with this method, an application device is loaded with bone growth composition or a combination of the bone growth composition and one or both of local autograft or bone marrow aspirate. The bone growth composition comprises: demineralized bone fibers generated from a donor bone tissue; cancellous chips and/or corticocancellous chips generated from the donor bone tissue; and a cryosolution comprising glycerol in Lactated Ringer's solution, wherein a glycerol content of the cryosolution is equal to or less than 25% by volume. A tip of the application device is positioned at an anatomic site of a patient. An actuation mechanism of the application device is operated to cause the bone growth composition or the combination of the bone growth composition and one or both of local autograft or bone marrow aspirate to be extruded from the tip of the application device at the anatomic site.

In a further embodiment, a mesh pouch is provided. In accordance with this embodiment, the mesh pouch comprises two layers of a mesh material secured along one or more edges so as to define an interior space and a plurality of projections configured to secure the two layers of mesh material along unsecured edges and allowing mesh pouch to be transitioned between an open and a closed configuration. The mesh pouch also comprises a volume of bone growth composition or a combination of the bone growth composition and one or both of local autograft or bone marrow aspirate positioned between the two layers and secured within the interior space, wherein the bone growth composition comprises: demineralized bone fibers generated from a donor bone tissue; cancellous and/or corticocancellous chips generated from the donor bone tissue; and a cryosolution comprising glycerol in Lactated Ringer's solution, wherein a glycerol content of the cryosolution is equal to or less than 25% by volume.

In accordance with aspects of the above embodiment, the two layers of the mesh material are secured using the plurality of projections so that one or both of a size or a shape of the mesh pouch conforms to an opening at an anatomic region. In accordance with other aspects of the above embodiment the mesh material comprises a woven, nonwoven, or knitted mesh material.

In a further embodiment, a pouch comprising a bone growth composition within a sealed and sterile environment is provided. In accordance with this embodiment, the pouch comprises: one or more drainage features that, when exposed, facilitate drainage of a cryosolution from the pouch. The bone growth composition comprises: demineralized bone fibers generated from a donor bone tissue; cancellous and/or corticocancellous chips generated from the donor bone tissue; and the cryosolution, wherein the cryosolution comprises glycerol in Lactated Ringer's solution, wherein a glycerol content of the cryosolution is equal to or less than 25% by volume.

In accordance with aspects of the above embodiment the pouch is formed integrally with an additional pouch such that the pouch and the additional pouch are in fluid communication when the drainage features are exposed. In accordance with certain additional embodiments the pouch further comprises a resealing feature configured to allow resealing of the pouch after opening.

In an additional embodiment, a method for providing bone growth composition at an anatomic region is provided. In accordance with this method, a volume of bone growth composition or a combination of the bone growth composition and one or both of local autograft or bone marrow aspirate is placed within an inner space defined by a mesh pouch. The bone growth composition comprises: demineralized bone fibers generated from a donor bone tissue; cancellous chips generated from the donor bone tissue; and a cryosolution comprising glycerol in Lactated Ringer's solution, wherein a glycerol content of the cryosolution is equal to or less than 25% by volume. One or more edges of the mesh pouch are secured using projections provided on one or more layers of mesh material used to form the mesh pouch. The mesh pouch is sized and/or shaped to conform to an anatomic region of a patient. The mesh pouch is inserted into the anatomic region.

### BRIEF DESCRIPTION OF THE DRAWINGS

Advantages of the disclosed techniques may become apparent upon reading the following detailed description and upon reference to the drawings in which:

FIG. 1 is a flow diagram of a processing method to produce a cellular bone matrix from donor bone tissue, in accordance with an embodiment of the present disclosure;

FIG. 2 shows an example bone region including sources for cancellous, cortical, and corticocancellous chips, in accordance with an embodiment of the present disclosure;

FIG. 3A shows an example bone region including sources for cancellous, cortical, and corticocancellous chips, in accordance with an embodiment of the present disclosure;

FIG. 3B shows an example bone region including sources for cancellous, cortical, and corticocancellous chips, in accordance with an embodiment of the present disclosure;

FIG. 4 is a flow diagram of a fiber processing method that may be used in conjunction with FIG. 1, in accordance with an embodiment of the present disclosure;

FIG. 5 shows example fibers, in accordance with an embodiment of the present disclosure;

FIG. 6 is a flow diagram of a two-step chip washing method, in accordance with an embodiment of the present disclosure;

FIG. 7 is a flow diagram of a two-step chip washing method, in accordance with an embodiment of the present disclosure;

FIG. 8 shows effects of the two-step chip washing method on a tissue sample relative to a control;

FIG. 9 is a flow diagram of chip washing, in accordance with an embodiment of the present disclosure;

FIG. 10 shows a comparison of cell viability for different washing techniques;

FIG. 11 shows a comparison of cell viability for different washing techniques;

FIG. 12 shows a comparison of cell viability for different cryosolutions;

FIG. 13 shows a comparison of cell viability for different cryosolutions;

FIG. 14 is a flow diagram of a method of contacting chips with a cryosolution for freezing, in accordance with an embodiment of the present disclosure;

FIG. 15 shows example components of a bone growth composition, in accordance with an embodiment of the present disclosure;

FIG. 16 shows an example bone growth composition packaged in a pouch and removed from an outer packaging, in accordance with an embodiment of the present disclosure;

FIG. 17 shows an example bone growth composition packaged in a pouch, in accordance with an embodiment of the present disclosure;

FIG. 18 shows an example bone growth composition packaged in a pouch and in a water bath for thawing, in accordance with an embodiment of the present disclosure;

FIG. 19 shows an example bone growth composition packaged in a pouch and being opened, in accordance with an embodiment of the present disclosure;

FIG. 20 depicts an example of a three-pouch embodiment for packaging, storing, and transporting bone growth composition, in accordance with an embodiment of the present disclosure;

FIG. 21 depicts the example of FIG. 20 in a use-configuration in which the inner pouch and pouch are nested within an outer pouch, in accordance with an embodiment of the present disclosure;

FIG. 22 shows an example of different conditions for a bone growth composition packaged in a pouch, in accordance with an embodiment of the present disclosure;

FIG. 23 shows an example bone growth composition packaging system including a release liner that is removed to permit draining, in accordance with an embodiment of the present disclosure;

FIG. 24 depicts an example of a pouch having integrated drainage features, in accordance with an embodiment of the present disclosure;

FIG. 25 depicts a further example of a pouch having integrated drainage features, in accordance with an embodiment of the present disclosure;

FIG. 26 is a flow diagram of assessing cell viability, in accordance with an embodiment of the present disclosure;

FIG. 27 shows a comparison of cell viability for different freezing techniques;

FIG. 28 shows a comparison of cell viability for different antimicrobial agents;

FIG. 29 shows a comparison of cell viability for different antimicrobial agents with respect to soak temperature;

FIG. 30 shows a comparison of cell viability for different antimicrobial agent concentrations;

FIG. 31 shows a comparison of cell viability for different antimicrobial agent rinse conditions;

FIG. 32 shows a comparison of post-thaw cell viabilities;

FIG. 33 shows a comparison of cell stability for different storage time conditions;

FIG. 34 depicts a first embodiment of an application device, in accordance with aspects of the present disclosure;

FIG. 35 depicts a second embodiment of an application device in a first configuration, in accordance with aspects of the present disclosure;

FIG. 36 depicts the second embodiment of the application device in a second configuration, in accordance with aspects of the present disclosure;

FIG. 37 depicts a third embodiment of an application device in a first configuration, in accordance with aspects of the present disclosure;

FIG. 38 depicts the third embodiment of the application device in a second configuration, in accordance with aspects of the present disclosure;

FIG. 39 depicts aspects of removing bone growth composition from a first packaging, in accordance with aspects of the present disclosure;

FIG. 40 depicts aspects of removing bone growth composition from a second packaging, in accordance with aspects of the present disclosure;

FIG. 41 depicts aspects of thawing bone growth composition, in accordance with aspects of the present disclosure;

FIG. 42 depicts aspects of thawing and handling bone growth composition, in accordance with aspects of the present disclosure;

FIG. 43 depicts aspects of removing bone growth composition from packaging, in accordance with aspects of the present disclosure;

FIG. 44 depicts further aspects of removing bone growth composition from packaging, in accordance with aspects of the present disclosure;

FIG. 45 depicts bone growth composition in a surgical tray prior to mixing with local autograft and/or bone marrow aspirate, in accordance with aspects of the present disclosure;

FIG. 46 depicts bone growth composition mixed with local autograft and/or bone marrow aspirate, in accordance with aspects of the present disclosure;

FIG. 47 depicts removal of a cannula tray from packaging, in accordance with aspects of the present disclosure;

FIG. 48 depicts removal of cannulas from a die card, in accordance with aspects of the present disclosure;

FIG. 49 depicts cannulas prior to loading with bone growth composition mixed with local autograft and/or bone marrow aspirate, in accordance with aspects of the present disclosure;

FIG. 50 depicts a cannula loaded with bone growth composition mixed with local autograft and/or bone marrow aspirate, in accordance with aspects of the present disclosure;

FIG. 51 depicts insertion of a loaded cannula into a delivery tube of an application device, in accordance with aspects of the present disclosure;

FIG. 52 depicts a cannula inserted into a delivery tube of an application device, in accordance with aspects of the present disclosure;

FIG. 53 depicts engagement of a delivery tube with an application device, in accordance with aspects of the present disclosure;

FIG. 54 depicts further aspects of engagement of a delivery tube with an application device, in accordance with aspects of the present disclosure;

FIG. 55 depicts additional aspects of engagement of a delivery tube with an application device, in accordance with aspects of the present disclosure; and

FIG. 56 depicts use of an application device to apply bone growth composition or bone growth composition mixed with local autograft and/or bone marrow aspirate at an anatomic site, in accordance with aspects of the present disclosure.

### DETAILED DESCRIPTION OF SPECIFIC EMBODIMENTS

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The methods discussed herein include various steps represented by blocks in flow diagrams. It should be noted that at least some steps may be performed as an automated procedure by one or more components of a system. Although the flow diagrams may illustrate the steps in a certain sequence, it should be understood that the steps may be performed in any suitable order and certain steps may be carried out simultaneously, where appropriate. Additionally, steps may be added to or omitted from of the methods.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements. One or more specific embodiments of the present embodiments described herein will be described below. In an effort to provide a concise description of these embodiments, all features of an actual implementation may not be described in the specification. It should be noted that in the development of any such actual implementation, as in any engineering or design project, numerous implementation-specific decisions must be made to achieve the developers' specific goals, such as compliance with system-related and business-related constraints, which may vary from one implementation to another. Moreover, it should be noted that such a development effort might be complex and time consuming, but would nevertheless be a routine undertaking of design, fabrication, and manufacture for those of ordinary skill having the benefit of this disclosure.

Composition, fabrication, use, and application of a bone growth composition are discussed herein. As discussed, the bone growth composition may comprise, in certain embodiments, a cellular bone matrix that may be formulated using human cryopreserved viable cortical cancellous bone matrix and demineralized bone fibers. As discussed herein, the disclosed bone growth composition can be used as a bone graft in orthopedic, reconstructive, or other suitable procedures and may be used in combination with autologous bone and/or bone marrow aspirate in appropriate use cases or may be used alone as a bone graft.

The disclosed bone graft compositions include demineralized allograft bone fibers from cortical bone, and cancellous and corticocancellous bone chips, and glycerol. The chips, being fully mineralized, retain their inherent radiopacity and enhance detection of the implant when using x-ray. The mineralized chips and demineralized fibers also impart desirable handling characteristics to the graft.

New bone formation in patients undergoing bone graft requires three elements; local signaling to drive new bone formation, a scaffolding material to template the shape of the future bone, and bone forming cells like osteoblasts and/or bone progenitor cells. The demineralized bone fibers expose local anabolic growth factors inherent in the tissue matrix. The demineralized fibers and cancellous bone chips also offer a scaffold material. In contrast to products in which the cells were provided by the recipient, the disclosed products provide allograft bone-forming cells.

The disclosed embodiments relate to bone growth compositions, such as bone matrix compositions. The bone matrix compositions may include cellular bone matrix compositions and techniques, also referred to as cellular bone matrices (CBMs) or viable bone matrices (VBMs). As provided herein, cellular bone matrixes provide a platform for new bone formation in a subject. A mix of bone elements that have live bone cells provides an osteoconductive scaffold and viable cells with osteogenic potential. Specifically, the CBMs provide various components for new formation, namely, an osteoconductive scaffold, extracellular growth factors for cell proliferation and differentiation, and viable cells with osteogenic potential. The growth factors and proteins in the demineralized fibers provide an active signal (i.e., osteoinductivity) for bone healing. Both the chips and fibers act as scaffolds for bone regeneration (i.e., osteoconductivity). Cellular bone matrices may be used for bone grafts, e.g., allogenic bone grafts. Cellular bone matrices include live bone cells, such as live mesenchymal stem cells (MSCs).

The CBM product is an autograft extender having osteoconductive, osteoinductive and osteogenic characteristics. The cryosolution, e.g., a glycerol and Lactated Ringer's solution (e.g., Lactated Ringer's), has been optimized to retain cellular viability. Also provided herein are effective manufacturing methods to optimize the manufacturing of this formulation in a way that maintains high cell viability, enhances microbiological safety, and enables high osteoinductive performance. These are described in more detail herein.

By design, the CBM is stored frozen using agents to maintain the viability of cells contained within the tissue. The tissue is prepared and packaged in a way that makes it ready-to-use following a short duration thawing process. The CBM can be utilized alone or mixed with autograft and/or bone marrow aspirate to enhance osteogenic characteristics and encourage bone fusion. The CBM may be moldable for insertion into a spinal implant cage or conforming to the contours of bone or other spinal region. Where the CBM is utilized for packing in a cage implant, the product may be compacted and malleable to conform to the designated space. In some instances, the composition can be dried by squeezing to alter its handling characteristics to the preference of the user. The composition alternatively be easily hydrated with fluid, water, blood, marrow, or otherwise to alter handling and increase malleability. Further hydration can enhance the compositions flowability for use with a graft insertion gun or funnel and enable it to be easily pressed through long cannulas less than about 8 mm in diameter. Once compacted or injected at the site for spinal repair, the graft is stable and unlikely to migrate due to the inherent cohesivity of the formulation. In some cases, the formulation is resistant to gentle irrigation.

Regarding appearance, the CBM has a fibrous appearance with particulate heterogeneity. In comparison with prior technology utilizing terminal sterilization, the embodiments herein comprise one or more steps of aseptic processing, variable according to batched donor supply and/or standardized for consistency across product line.

The improved thawing characteristics of the CBM include flexible packaging to permit an expanded surface area exposed to thawing temperatures and minimization of air pockets that inhibit thawing.

The bone repair donor materials disclosed herein may be autologous, allogenic, or xenogenic. For example, an autograft, as used herein, may refer to bone and/or tissue that is extracted from the intended recipient of an implant. An allograft refers to bone and/or tissue that is from a donor that is different than the intended recipient. A xenograft refers to bone and/or tissue that is from a donor that is a different species than the intended recipient. The disclosed compositions and techniques may be used in the context of autologous, allogenic, or xenogenic compositions or mixtures thereof. Further, the disclosed products may be formed from one or more donors, e.g., a single product may be formed from a single donor or from donor materials from multiple donors. For example, in certain cases, an allogenic material may be mixed with an autograft material and/or bone marrow aspirate to form a final product that is provided to the patient. The donor bone tissue may be cadaveric or from a living donor. In an embodiment, the bone growth composition uses tissue originating from a recently deceased human and is processed within a sufficiently short timeframe to enable retention of cellular components. The donor bone tissue may be a bone end of a long bone, from bone locations proximal to joints, or within the interior of vertebrae. By way of example, the donor bone tissue may be a distal end radius, proximal humerus, or proximal femur.

In embodiments, tissue recovered for use in the CBM product will be from donors screened, recovered, and tested in compliance with current AATB and FDA guidance documents. In an example, the source bone will originate anatomically from the extremities and from portions of the pelvis. In an embodiment, bone originating from the spine, ribs, cranial region, hand, or foot anatomy is not used. The disclosed embodiments may include testing and exclusion of any donor that tests positive for HTLV I/II. Donor tissue which tests positive for Streptococcus pyogenes, Clostridium sp, or Mycobacterium tuberculosis organisms (by PCR and/or culture) may also be excluded.

In an embodiment, the donor tissue is in compliance with AATB bulletins 22-2 and 23-6. These documents make additional recommendations for tissue banks to consider to limit the risk of Mycobacterium tuberculosis transmission. The CBM products may exclude tissue from donors with a history of dialysis outside of an ICU setting or donor with a history of long-term steroid use prior to death.

Donor tissue is recovered within the time period defined by AATB guidelines. Following recovery, tissue is stored and transported to on wet ice or other appropriate conditions per AATB guidelines. In an embodiment, donor tissue may not be frozen prior to processing into the bone composition product. In an embodiment, donor tissue must complete its processing into the finished product within 96-120 hours of donor time of death or, in an embodiment, within 96 hours of donor time of death.

For clarification, viable cells within CBMs are cell populations capable of promoting synthesis of new bone, such as multipotent adult progenitor cells (MAPC), mesenchymal stem cells (MSCs), osteoprogenitor cells (OPCs), osteocytes, and/or osteoblasts (OBs). MAPCs and MSCs are both non-hematopoietic cells found in the bone marrow stroma; these cells retain the ability to self-replicate and differentiate into a specific phonotype by intrinsic and local environmental cues (e.g., spatial organization, mechanical forces, growth factors). Many other cells common to bone tissue may also be present in CBMs. Those could include fibroblasts, adipocytes, vascular endothelial cells, hematopoietic cells, lymphocytes, and other cells found commonly in bone.

In certain embodiments, techniques for preparing, manufacturing, storing, and/or applying cellular bone matrix compositions are provided. In certain embodiments of the invention the disclosed techniques permit improved biological activity (e.g., osteoinductivity and/or osteoconductivity) of cellular bone matrices at one or more preparation stages. The cellular allografts, CBM/VBMs, are obtained by processing to largely remove immune-responsive signals generated by bone marrow components. Such components could include hematopoietic cells, retaining bone-forming cells within the bone matrix.

Embodiments of the disclosed techniques relate to cellular bone matrix processing methods that include an antimicrobial agent contact step. Use of an antimicrobial agent during processing may improve clinical results and end product characteristics by reducing potential for spoilage and improving shelf life of the cellular bone matrix. This contact step may also be used to reduce or eliminate microbiological contamination originating from the starting tissue or manufacturing process itself. However, the antimicrobial agent contact during processing may reduce cell viability for active cells of the chips. Certain antimicrobial contact conditions as disclosed herein may be associated with relatively higher cell viability. In addition, the disclosed techniques permit inclusion of an antimicrobial contact step in cancellous chip processing by maintaining or improving cell viability at other steps in chip processing, such that cell loss or reduced viability as a result of the antimicrobial contact step is compensated for by improved cell viability at other stages. These high viabilities in the manufacturing work flow result in a finished product with high cellular viability even while including aggressive antimicrobial steps to minimize the potential for bacterial and/or fungal contamination in the finished product while maintaining high performance.

In particular, cryosolutions that permit storage of the cellular bone matrix products may nonetheless have harmful effects on cell viability. Embodiments of the disclosed techniques include cold preservation and cryopreservation in cryosolution formulations and conditions with associated improved cell viability characteristics. By way of example, such a cryosolution may be employed for tissue storage, transport, washes or rinses, and so forth. Such a cryosolution may further be utilized in temperatures equal to and greater than −80° C. and/or may be used in various storage or use contexts to preserve a higher percentage of osteoblasts than can normally be preserved using conventional approaches and formulations. By way of example, osteoblasts, which are typically present in the mineralized portion of the bone, may have a higher survival rate using the techniques and cryosolution discussed herein.

The disclosed techniques also include cellular bone matrix processing with wash or rinsing step conditions with unexpected cell viability benefits relative to conventional processing steps. In embodiments, the disclosed techniques include contacting a cancellous portion with glycerol combined with Lactated Ringer's at glycerol concentrations that, for example, provide enhanced cell viability at one or more freezing temperature conditions. In certain cases, the disclosed product can be maintained for two or more months (e.g., six months, nine months, twelve months, or more) at −70 to −80° C. storage conditions. Such considerations may be of significance in terms of real-world implementations. In particular, storage units and/or freezers capable of operating at −80° C. and 70° C. m cost in the range of $15,000 to $30,000 and up, which may be a substantial investment. Further, in practice cell viability may be impacted when cellular material is maintained at temperatures below −80° C. and viability of cells that are stored in conventional solutions (e.g., DMSO) may be detrimentally impacted. Further, such conventional solutions (e.g., DMSO) must be rinsed off and removed from transplant materials prior to use. With this in mind, cryosolution formulations as discussed herein may allow for the use of more readily available and less expensive storage and refrigeration options, such as +4° C. refrigerators used for chromatography supplies, blood storage, and pharmaceuticals, −20° C. (and below) freezers used for storing enzymes and biochemicals: −30° C. to −40° C. storage used for biological samples, and/or (iii) −80° C. freezers for long term storage and stability. As may be appreciated, price ranges for these options may vary drastically. Further, kitchen units (e.g., non-medical grade units available at low cost) can be used for low risk materials.

Although popular among surgeons, many cellular bone matrices have complicated thawing and preparation steps. For example, thawing in a jar or rigid container can take up to 30 minutes. In contrast, in an embodiment, the disclosed cellular bone matrix compositions are frozen in flexible bags, flexible pouches, vessels, or receptacles such as a pouch. The CBM is frozen in pouches as the primary packaging which permits much faster thawing (<15 minutes). This facilitates access and quick access in a surgical setting. In one aspect, the malleable pouch allows for external manipulation of the CBM as it thaws, such as via an application device configured to receive such a pouch (or other comparable container) and allow or facilitate directed application of the thawed CBM. By way of example, in one implementation the CBM may be thawed in a sealed or perforated (e.g., suitable for tearing open along the perforation) pouch or bag that remains in the preservative solution while thawing. In practice, the preservative solution may be frozen prior to thawing and when thawing begins, with thawing occurring as quickly as practical to preserve cell viability. In certain embodiments, a multi- or dual-pouch solution (e.g., an inner pouch containing the CBM and a main pouch in which the inner pouch is disposed) may be provided. In one such implementation, the main pouch may include a rubberized seal which may be used to insert liquid cryosolution to moderate or accelerate the thawing process.

In addition, many cellular bone matrices require that the cryosolution be poured/decanted off after thawing because of dimethyl sulfoxide (DMSO) toxicity. Sometimes, the tissue has to be rinsed with saline. In contrast, the disclosed cellular bone matrix compositions do not require decanting or washing to maintain high cell viability after thawing. If, however, the user decants off any volume of liquid or storage solution, the pouch can be pinched off at the end to facilitate decanting. As well, a modified end portion of the pouch may incorporate a perforated sheet integrated with the sidewalls of the pouch to contain the CBM while allowing the extra volume of liquid to be easily extracted or poured off. As well, CBM packaged within individual cannulas internal to the pouch will be capable of being frozen and thawed from a freezing state in reduced times compared to that of ajar. These features enhance end user workflow relative to products that involve more complex handling.

A cellular bone matrix composition may be formed from a cancellous portion of a bone (e.g., cancellous and/or corticocancellous chips) and a noncancellous, e.g., cortical or compact, portion of the bone (e.g., cortical fibers). One embodiment of the noncancellous portion of bone includes cortical bone particles, pieces, segments, and/or fibers. When harvesting or grating fibers from the donor tissue, the fibers are produced in a ribbon-like configuration, the fibers of which are irregular in shape with expansive surface areas. By way of example, in certain embodiments surface are for such bone fibers is greater than 1,000,000 cm²/g (e.g., 1,014,895 cm²/g or 1,082,011 cm²/g). For exemplary purposes, and not limitation, the ribbon-like structures maintain the ribbon-like configuration that is a columnar-like sheet that wraps around itself. The ribbon-like configuration has increased surface area via the curled portion and, in embodiments, surface nanostructures. In some embodiments, nanostructures are imparted to bone matrix compositions wherein the nanofibrous properties on the surface may be compromised. In other embodiments, the bone is processed in a manner so as to expose and/or maintain the nanofibrous structure of the bone. The nanostructures may be as disclosed in U.S. Pat. No. 10,220,115, the disclosure of which is hereby incorporated by reference in its entirety herein for all purposes.

The particulates or fibers harvested range in size with variations from millimeters to centimeters, as described herein. At the end stage of milling, the particulates and fibers may range in size to form a non-homogenous fiber mix. In an embodiment, the fibers may range in micron sizes from about 50 microns to 5 cm or 10 cm. In an embodiment, the noncancellous portion contains less than 10% soft tissue. Both the chips and fibers, when provided as a cellular bone matrix, act as a scaffold for bone formation to a subject in need of bone repair. The cancellous portion, which contains viable bone cells, is processed separately from the noncancellous portion during cellular bone matrix manufacturing to retain the viability of the bone cells during processing. That is, processing steps applicable to the cortical fibers may be unsuitably harsh for the cancellous chips. However, certain processing steps to clean, disinfect, or cryopreserve the cancellous portion may nonetheless reduce bone cell viability. In an embodiment, the disclosed techniques permit processing of the cancellous portion to clean, disinfect, and/or cryopreserve while maintaining adequate bone cell viability. Thus, the cellular bone matrix end product produced by the disclosed techniques has desirable clinical characteristics.

The mix of chips and demineralized fibers in cellular bone matrix compositions as provided herein provide excellent handling. The fibers yield a cohesive putty-like implant, and the mineralized chips provide some grittiness. Internal testing has shown that the disclosed cellular bone matrix compositions are osteoinductive in the gold standard athymic rat muscle pouch model.

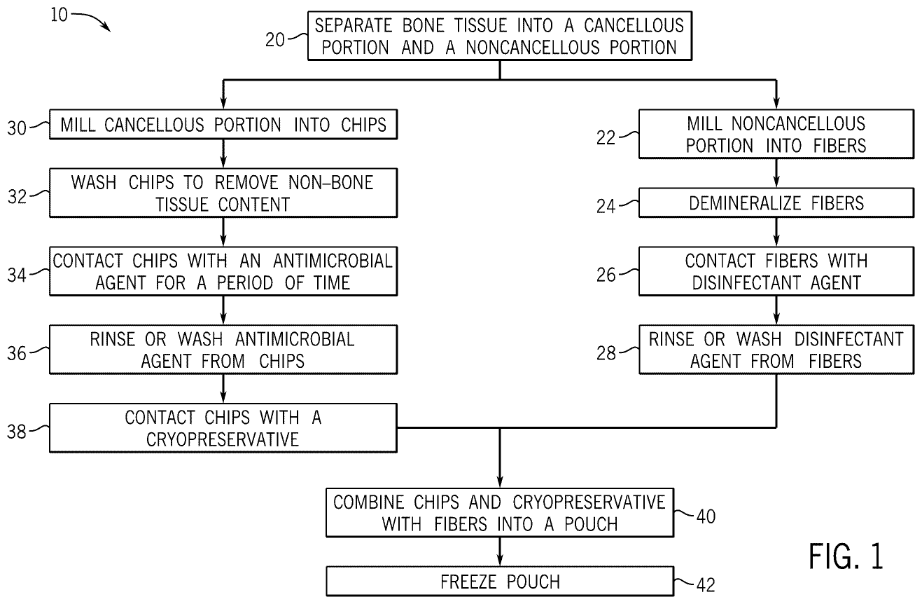

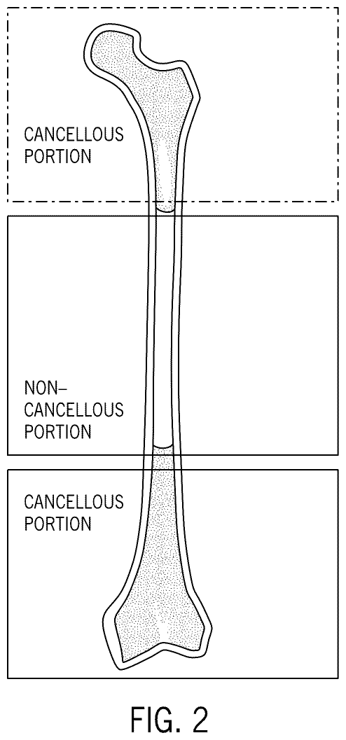

FIG. 1 is a processing method 10 to produce a cellular bone matrix from donor bone tissue. At step 20, the donor bone tissue is separated into a cancellous portion and a noncancellous portion, as shown by way of example in FIG. 2. In an embodiment, the donor bone tissue exterior is cleaned. Donor bone treatment may also include a debridement step to remove non-bone tissue content prior to separation. In one aspect, a high-pressure water cleaning process is used to clean the bone. In embodiments, the donor tissue is provided without having been previously frozen to maintain cell viability. Thus, the disclosed bone growth compositions, after freezing, may be considered fresh-frozen. The separation into the cancellous portion and the noncancellous portion may be via cutting or sawing techniques to remove cancellous tissue from the distal and proximal ends of a bone shaft. The cancellous portion and the noncancellous portion may be cut or segmented during or after separation into blocks that are suitably-sized for downstream steps of the process 10.

As is often the case, noncancellous bone is attached to or encasing or surrounding the cancellous bone elements of the tissue being segmented from the native anatomy. Thus, as provided herein, the cancellous portion may also include noncancellous elements in some nonzero percentage that are incorporated and retained within the subsequent chip form. Further, as provided herein, the noncancellous portion and the cancellous portion may not be achieved with 100% homogeneity. In fact, this imprecise segmentation is desirable as it minimizes donor material loss by eliminating unnecessary cutting and trimming of the tissue during segmentation. It should be understood that the cancellous and noncancellous portions of bone as described herein are predominately cancellous bone or noncancellous bone, but comprise some elements of both. In an example, the cancellous portion and the noncancellous portion may be cut into blocks that are between 3-10 cm in one dimension. Processing may be modified here to cut at a desired size. As well, the tools utilized for creating the particulates or fibers may determine the portion sizes created.

Turning to the processing of the noncancellous portion as discussed in FIG. 1, the noncancellous portion, at step 22, is milled or otherwise processed (e.g., cut, grated, pressed, shaved, shredded) into fibers. The fibers may be any suitable size or shape. In an embodiment, the fibers have a length dimension that is at least twice a width and height dimension. In an embodiment, the fibers have an aspect ratio of at least 50 (length):1 (height and/or width), and an average length greater than 0.5 cm. In an embodiment, the width and height dimension of the fiber have about a 1:1 aspect ratio while the length dimension is elongated. In an embodiment, a fiber length dimension is at least 5, 100, or 1000 times a width and height dimension. Example ranges of a length dimension relative to other dimensions are between 5-50 times, 5-100 times, or 5-1000 times. In an embodiment, the fibers may be uniform or nonuniform, e.g., may have a uniform or nonuniform length, width, and or surface area. In an embodiment, the fibers may be curled or ribbon-like. In an embodiment, the fibers are less than 10 or 5 mm in any dimension. In some embodiments, the bone fibers have a diameter from about 100 m to about 2 mm. In some embodiments, the bone fibers have a length from about 0.5 mm to about 50 mm. In some embodiments, the bone fibers have an average length from about 0.5 cm to about 10 cm. In an embodiment, the average bone fiber thickness is between 0.05 to 0.5 mm.

In one embodiment, the bone fibers are milled using an osteobiologic milling machine as disclosed in U.S. Pat. No. 9,004,384, the disclosure of which is hereby incorporated by reference in its entirety for all purposes. The osteobiologic milling machine may be computer-controlled, with instructions that are programmed, stored, and/or executed by a programmable logic controller. The instruction may control milling parameters such as fiber length, thickness, and/or surface area. In an embodiment, the output of the osteobiologic milling machine may include material that is outside of tolerance. Thus, in an embodiment, the output may undergo a sorting or sieving step to remove bone components that are too long, too short, too thin, or too thick.

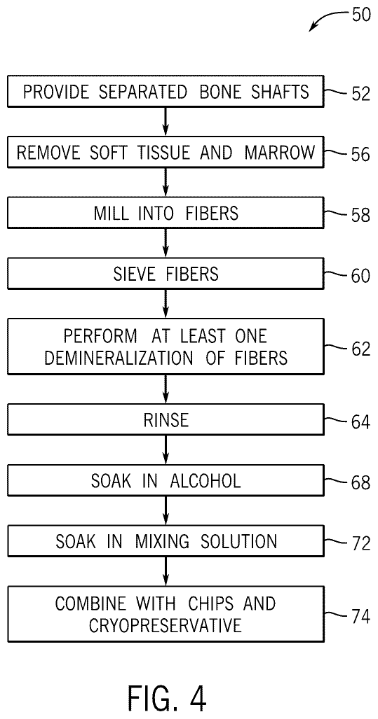

At step 24, all or some of the fibers are demineralized, which may refer to a process of removing inorganic content, such as minerals, from cortical fibers. The fiber end product after demineralization may have at least 50% or at least 90% or at least 99% of minerals (e.g., calcium) removed. In an embodiment, the fibers may be demineralized such that the fiber end product after demineralization has less than 1% residual calcium. Demineralization may occur via an acid soak (e.g., HCl, 0.6N HCl). At step 26, the demineralized fibers are disinfected with an alcohol solution (70% alcohol in water by volume) and at step 28 rinsed/transferred to a solution, such as Lactated Ringer's, to soak before being combined with the processed cancellous portion at step 40. An embodiment of cortical fiber processing is discussed in more detail in FIG. 4.

Demineralized bone matrix (DBM) has been shown to exhibit the ability to induce and/or conduct the formation of bone. Demineralization improves flexibility and handling characteristics. Also, calcium/mineral phase of bone can cover the collagen phase of bone. Demineralization exposes the bone growth factors to facilitate bone growth. It is therefore desirable to implant and maintain demineralized bone matrix at a site which bone growth is desired. Bone fiber based-demineralized bone matrices for implantation exhibit improvements in mechanical properties, including cohesiveness, fiber length, fiber diameter or width, fiber aspect ratio, or a combination of multiple variables.

Turning to the processing of the cancellous portion, at step 30, the cancellous portion removed from the bone tissue is milled into chips. As disclosed, the cancellous portion may refer to a bone tissue portion that is majority or predominantly cancellous tissue (e.g., greater than 50%, greater than 75%, or greater than 90% by volume) but that nonetheless includes other elements, such as some encasing cortical tissue as well as marrow elements, blood, fat that remain before cleaning. Indeed, a benefit of the disclosed technique is that milling the cancellous bone with the marrow elements present protects the cells within the tissue to improve viability and/or cell retention at this stage. By way of example, in certain implementations cell marker CD45 (which is expressed by immunogenic white blood cells) was detected in the milled bone tissue. In particular, such markers and/or immunogenic cells were observed at levels approximately 10× higher than was observed for a cell-free bone chip control sample included for comparison.

While fibers may be characterized by an elongated form, a chip in certain embodiments may have a planar form having a width dimension that is greater than a width of a fiber and a length dimension that is less than a fiber length while having a similar height dimension. In an embodiment, a chip aspect ratio is at least 50:1, and the chip an average length greater than 0.1 cm (e.g., 0.5 mm-2 mm). In an embodiment, the chips may be uniform or nonuniform. In an embodiment, the chips are less than 5 mm in any dimension.

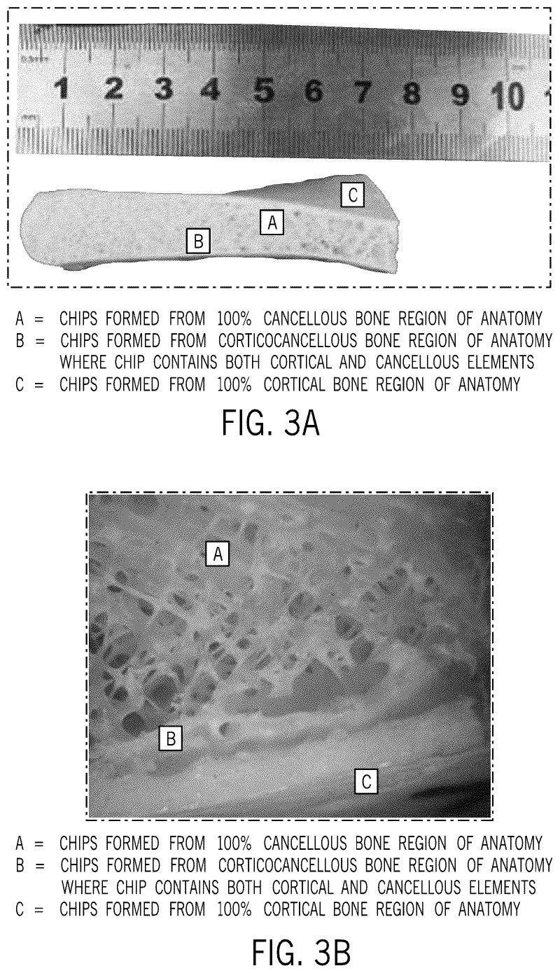

FIGS. 3A-B show an example cancellous portion of a bone tissue (see FIG. 2), e.g., a femur end, that is processed to form chips and that is predominantly cancellous tissue but that includes example cancellous (A), corticocancellous (B), and cortical (C) bone regions. The cancellous, cortical, and corticocancellous bone regions are respective sources for cancellous, cortical, and corticocancellous chips. As discussed, the population of chips is predominantly cancellous (e.g., greater than 50%, greater than 75%, greater than 95%) by percentage. However, a certain percentage of chips include cortical or noncancellous tissue. Further, an individual chip of the chips may be comprised of 100% cancellous tissue, 100% cortical tissue, or may be a corticocancellous chip generated from a corticocancellous region, e.g., a heterogenous mixed chip that includes both cancellous tissue and cortical tissue present on a single chip. In one example, cancellous chips may include a population of chips are 50-99% cancellous with the remaining chips being one or more of noncancellous or mixed cancellous/noncancellous. In one example, the chips are about 80-90% cancellous and about 10-20% mixed cancellous/noncancellous. In one example, the chips are about 90-99% cancellous and about 1-9% mixed cancellous/noncancellous. The cancellous chips as provided herein may refer to a set or population of chips that is at least 50% cancellous. Cortical chips may be less porous and more dense than cancellous chips. Thus, while the chips may be sized and shaped similarly, their structural qualities may be different. In an embodiment, cancellous chips have at least 1% noncancellous materials. Further, demineralized bone fibers formed from the cortical portion may also have a heterogenous composition that includes at least 1% noncortical materials.

The chips are washed at step 32 to begin removal of non-bone tissue elements and contacted with an antimicrobial and/or antifungal agent(s) at step 34. The chips may be soaked in the antimicrobial and/or antifungal agent(s) for a period of time, e.g., a predetermined period of time. In an embodiment, the antimicrobial and/or antifungal soak is conducted in a temperature range of 25-37° C. and for at least 1 hr or in a range of 3 hrs-12 hrs. In an embodiment, the antimicrobial and/or antifungal soak is conducted at 35-37° C. or 37° C. and for at least 5 hours but no more than 10 hours. The antimicrobial and/or antifungal solution is selected based on characteristics of the antimicrobial agent and antifungal agent and at a concentration sufficiently high to kill undesirable microorganisms or fungal elements. In an embodiment, the antimicrobial and/or antifungal agent is mixed with water (e.g., filtered water). In an embodiment, the antimicrobial and/or antifungal agent is mixed with an isotonic solution (e.g., Lactated Ringer's solution, saline, etc.). In an embodiment, the soak is conducted with agitation and/or stirring.

After the antimicrobial and/or antifungal contact, the chips are washed to remove the antimicrobial and/or antifungal agent (or to leave only residual amounts of the antimicrobial and/or antifungal agent) using one or more washes at step 36. The wash step can use Lactated Ringer's (e.g., Lactated Ringer's solution or Ringer's lactate) to facilitate transition to the cryosolution contact at step 38. In certain embodiments, the wash or rinse is conducted multiple times, e.g., three times, with removal of rinse solution between the rinses. In certain embodiments, the wash or rinse is timed (e.g., 5-15 minutes), and includes agitation (e.g., light stirring, etc.). After the antimicrobial contact, the chips are antimicrobial-treated chips. At step 38, the antimicrobial-treated chips are soaked in a cryosolution. In an embodiment, the cryosolution is 10-25% glycerol by volume and 75-90% mixing solution. In an embodiment, the cryosolution is 15% glycerol by volume, 85% mixing solution. In an embodiment, the mixing solution is Lactated Ringer's. In an embodiment, the cryosolution is 20% glycerol (e.g., has a glycerol content of 20% by volume), 80% Lactated Ringer's. In an embodiment, the chips are soaked in the cryosolution for a period of time, e.g., a predetermined period of time. In an embodiment, the chips are soaked in the cryosolution for at least 30 minutes, at least 60 minutes, at least 120 minutes, or at least 180 minutes. In an embodiment, the chips are soaked in the cryosolution for 30 minutes to 240 minutes. In an embodiment, the chips are soaked in the cryosolution for 60 minutes to 180 minutes. The cryosolution can be provided in sufficient volume to cover the chips. In certain embodiments, after completion of the soak, at least some of the cryosolution can be decanted or removed in preparation for freezing.

In an embodiment the Lactated Ringer's as provided herein may be Lactated Ringer's (e.g., Baxter Lactated Ringer's from Baxter, B. Braun Lactated Ringer's from B. Braun Medical Inc.) having the following composition: each 100 mL of Lactated Ringer's contains: Sodium chloride 600 mg; sodium lactate, anhydrous 310 mg; potassium chloride 30 mg; calcium chloride, dihydrate 20 mg. The pH is 6.6 (6.0-7.5). One liter has an ionic concentration of 130 mEq sodium, 4 mEq potassium, 2.7 mEq calcium, 109 mEq chloride and 28 mEq lactate. The osmolarity is 525 mOsmol/L (calc).

In certain embodiments, the cryosolution is glycerol, and the cryosolution does not include DMSO. In an embodiment, cryosolution is glycerol, and the cryosolution does not include any additional cryosolution agents. This cryosolution in combination with the viable bone cells and/or demineralized fibers may be referred to in embodiments as a cryo-ready product or bone growth composition.

At step 40, the chips and fibers are combined for storage. As discussed above, the chips are processed prior to freezing and complete processing wetted with cryosolution. The fibers are processed and may complete processing with a soak or wash in the mixing solution without any cryosolution, e.g., in a Lactated Ringer's solution. Thus, mixing of the chips and fibers dilutes the existing cryosolution present with the chips. The amount of dilution depends on 1) the volume of Lactated Ringer's solution carried together with the fibers relative to the volume of cryosolution carried together with the chips at combination and 2) the ratio of chips to fibers.

In an embodiment, the ratio of chips to fibers is 1:1. In some embodiments, the chips to fibers ratio is about 90:10, 80:20, 75:25, 70:30, 60:40, 50:50, 40:60, 30:70, 25:75, 20:80 and/or 10:90. In some embodiments, the chips to fibers ratio is in a range between 70:30 and 30:70, between 60:40 and 40:60, between 55:45 and 45:55, or between 51:49 and 49:51. The ratio can be assessed as a volume:volume or a weight:weight ratio.

After combination, the combined chips and fibers that form the cryo-ready product are sealed in an appropriate freezing container, e.g., a pouch, and exposed to a chilled environment, a cold environment, or a freezing environment at step 42. In embodiments, the cold environment is a refrigerator temperature that is 5° C. or less and/or between 0° C. and 5° C. In embodiments, the freezing environment is a cryogenic environment. In an embodiment, the freezing environment is 0° C. or less. In an embodiment, the freezing environment is 0° C. to −200° C. In an embodiment, the freezing environment is 0° C. to −20° C. In an embodiment, the freezing environment is −15° C. to −20° C. In an embodiment, the freezing environment is −15° C. to −100° C. In an embodiment, the freezing environment is −15° C. to −80° C. In an embodiment, the freezing environment is −40° C. to −80° C. In an embodiment, the freezing environment is −65° C. to −80° C. In an embodiment, the freezing environment is −70° C. +/−15° C. In an embodiment, the freezing environment is −80° C. +/−15° C.

In certain embodiments, the disclosed cryo-ready product provides improved stability and viability even if certain steps in the freezing, storage, transport, and/or thawing process are not performed according to recommended guidelines. Further, the disclosed cryo-ready product, when frozen or chilled, may maintain viability even during conditions in which enzymatic or other chemical processes of the viable cells are still active at a lower level based on the storage temperature. In an embodiment, the disclosed cryo-ready product may be stored at temperatures at which certain cell processes, such as enzymatic reactions, are not completely shut down.

FIG. 4 is an example fiber processing method 50 that may be used in conjunction with FIG. 1. At step 52, bone shafts separated from bone ends are provided, and soft tissue and marrow is removed at step 56. At step 58, bone shafts, which may be provided as segments (e.g., 50-150 mm long segments) are milled into fibers. The milling may be performed using a cartridge mill. The fibers are sieved and shard material can be removed during the process at step 60. The fibers undergo at least one and, in certain embodiments at least two demineralizations at step 62. The demineralization may be an acid soak, such as a 0.6N HCl soak. In an embodiment, the first demineralization is a 15-150 minute acid soak with occasional stirring. In an embodiment, the second demineralization is a 45-260 minute acid soak with occasional stirring. During acid soaks, pH may be monitored with a target pH of less than 1. Soak time or other conditions can be adjusted to achieve the target pH.

The fibers are rinsed in water (e.g., filtered water) at step 64. In an embodiment, the rinse is conducted at least two or three times (e.g., ten minute soaks with soak solution replacement), and the fibers are subsequently soaked in alcohol at step 68. The alcohol soak may be in 70% ethanol (e.g., 20-200 minutes). The alcohol soak is followed by a soak in the mixing solution used for the cryosolution, e.g., Lactated Ringer's at step 72 until the measured pH is at least 3 (e.g., 5-800 minutes). The demineralized fibers are then combined with chips and cryosolution at step 74. FIG. 5 shows example fibers.

In some embodiments, the bone fibers have an average length to average thickness ratio or aspect ratio of the fibers from about 50:1 to about 1000:1. In overall appearance the bone fibers can be in the form of ribbons, threads, narrow strips, and/or thin sheets. The elongated bone fibers can be substantially linear in appearance or they can be coiled to resemble springs. In some embodiments, the bone fibers have linear portions and coiled portions. In some embodiments, the bone fibers are of irregular shapes including, for example, linear, serpentine and/or curved shapes. In some embodiments, the fibers can be curled at the edges to have substantially hemicircular cross-sections. In some embodiments, the fibers may be entirely or partially helical, circumvoluted or in the shape of a corkscrew. The elongated bone fibers can be demineralized however some of the original mineral content may be retained when desirable for a particular embodiment or within regulatory or process-specific tolerances (e.g., <6%, <2%, <1%, and so forth). The bone graft fiber may further comprise mineralized bone material.

The bone fibers may be elongated and curled to increase the surface area of the strips. The curled fibers may include frayed portions along the edges to facilitate interactions with other bone fibers. In some embodiments, the curled fibers are milled to have hooked portions along the edges of the fibers configured to engage with other fibers. The hooked portions may engage other hooked portions, frayed portions, straightened portions or curled portions of other fibers. The hooked and frayed portions and the curled shape of the fibers provide for entanglement between fibers such that the fibers may form a coherent mass without the need for a carrier or binding agent, as generally disclosed in U.S. Patent Publication No. US20210402060A1, the disclosure of which is incorporated by reference in its entirety and for all purposes herein. In addition, aspects and teachings of U.S. Pat. Nos. 5,314,476 and 5,676,146, which relate to the use of demineralized bone particles in an osteogenic composition and to the use of radiopaque markers in implants respectively, are incorporated by reference in their entirety and for all purposes herein. The fibers may include nanofibers of a submicron level or any fibers having at least one side or dimension at or below 1000 nanometers. In specific embodiments, the fibers may have at least one side or dimension at or below 100 nanometers. The fibers may include nanostructures, such as nanofibers, nanoparticles, nanospheres, nanopores, nanomicelles, and nano-roughness on surfaces. Nanostructures include structures ranging from approximately 1 nm to approximately 1000 nm in at least one dimension. The nanostructures may be part of a nano-textured surface.

In an embodiment, the bone growth composition materials, such as the fibers or chips, may include nano-scale textured surfaces attractive to cells. Nano-scale textured surfaces provided on a bone matrix aid in growth factor retention, remodeling, cell attachment, and osteoconductivity of the bone matrix. In embodiments wherein the nanostructures of the textured surface comprise nanofibers, the nanofibers may be oriented. In some embodiments, the nanostructures of the textured surface may be biologically active. For example, the nanostructure may comprise biologically active biomolecules or incorporated with other biological factors such as peptides, growth factors, cytokines, DNA, RNA, siRNA etc.

In an embodiment, the nanoscale textured surface of bone is retained during processing steps to prepare the bone growth composition. In an embodiment, the nanoscale textured surfaces are imparted to bone or enhanced via one or more processing steps. In certain cases, the fibers are prepared without lyophilization or drying. Because removal of moisture can deteriorate surface nanostructures, maintaining wetted fibers as provided herein may be associated with improved nanoscale feature retention. However, in embodiments, even in cases where the fibers and/or chips remain wet through the process, the bone growth composition may be subjected to steps that improve nanoscale features on the bone surface, such as providing hydrogel carriers (e.g., dextran, pluronics, N,O-carboxymethylchitosan glucosamine) and/or adding nanoscale features as a surface coating or treatment via electrospinning nanofibers (e.g., collagen, polylactide, polycaprolactone, polyglycolide, chitosan, gelatin, or other nanofibrous material) onto bone surface. The nanofibers may include a pharmaceutical agent or bioactive material as provided herein.

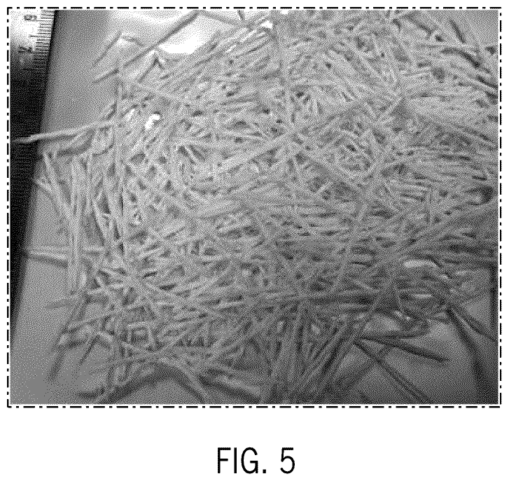

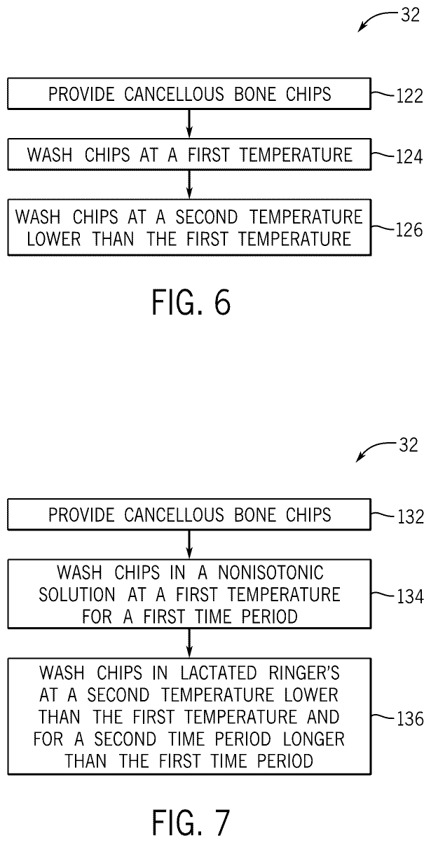

FIG. 6 is a two-step chip washing process 32 that may be used in conjunction with FIG. 1 or other bone handling processes. In an embodiment, the chip washing process 32 may be performed after milling a cancellous portion into chips. At step 122, cancellous bone chips are provided (e.g., from milling as in FIG. 1), and the chips are washed at a first temperature at step 124 and subsequently washed at step 126 at a second temperature lower than the first temperature. The first wash may be in the same or a different solution than the second wash.

FIG. 7 is an embodiment of the two-step chip washing process 32 of FIG. 4. At step 132, cancellous bone chips are provided (e.g., from milling as in FIG. 1), and the chips are washed in a first washing solution that is a nonisotonic solution (e.g., water, filtered water) at a first temperature for a first time period at step 134 and subsequently at step 136 washed in a second washing solution that is Lactated Ringer's at a second temperature lower than the first temperature and for a second time period longer than the first time period (e.g., the first time period is shorter than the second time period). The illustrated process 32 is by way of example, and the time of washes may, in embodiments, be the same (e.g., 5-15 minutes), or the second wash may be shorter in time than the first wash in another embodiment.

In an embodiment, the first wash step is conducted at 40-50° C. (e.g., 42-44° C.) for 5-15 minutes in a closed vessel with agitation, spinning, and/or a motorized impeller. In an embodiment, the first wash step is conducted for a minimum of 5 minutes and a maximum of 15 minutes. The second wash may be conducted at 22-35° C. (e.g., 25° C.) for 5-20 minutes or at least 20 minutes in a closed vessel with agitation, spinning, and/or a motorized impeller.

The water used in the first wash is nonisotonic relative to the bone cells of the chips. The two-step wash maintains cell viability relative to a wash in an isotonic solution, which is an unexpected benefit.

FIG. 8 shows a side-by-side comparison of a cancellous tissue portion sample 150 and associated histology image 160 washed with the two-step wash as discussed in FIG. 5 relative to a control cancellous tissue portion sample 152 and associated histology image 162 processed in a one-step wash. As shown in the image 160, the sample 150 has limited marrow and soft tissue attachment to chips while the image 162 is indicative of marrow and/or soft tissue attached to most chips in the control sample 152. The effectiveness of the two-step chip washing is demonstrated. Using this technique, there is very little marrow and soft tissue still attached to the bone chips after cleaning (left image 160). Very small infrequent pockets of non-bone tissue (circled in left image 160) can only be found at high magnification. When a single-step wash technique is used, considerable bone marrow and soft tissue still remain (circled in right image 162). Large pockets of non-bone tissue are evident throughout even at low magnification.

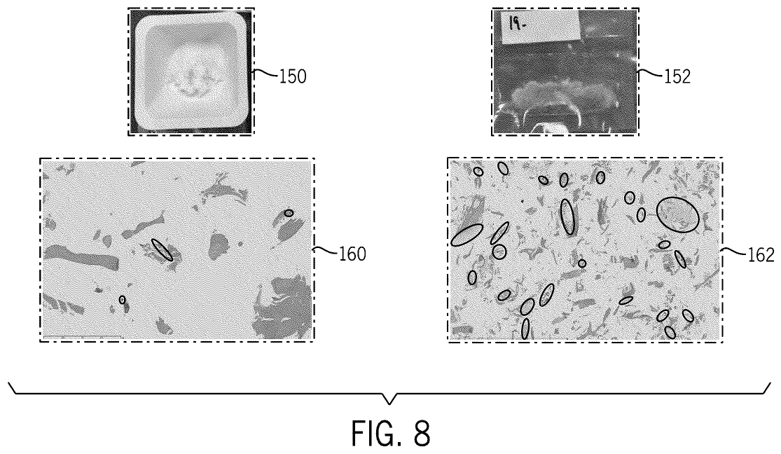

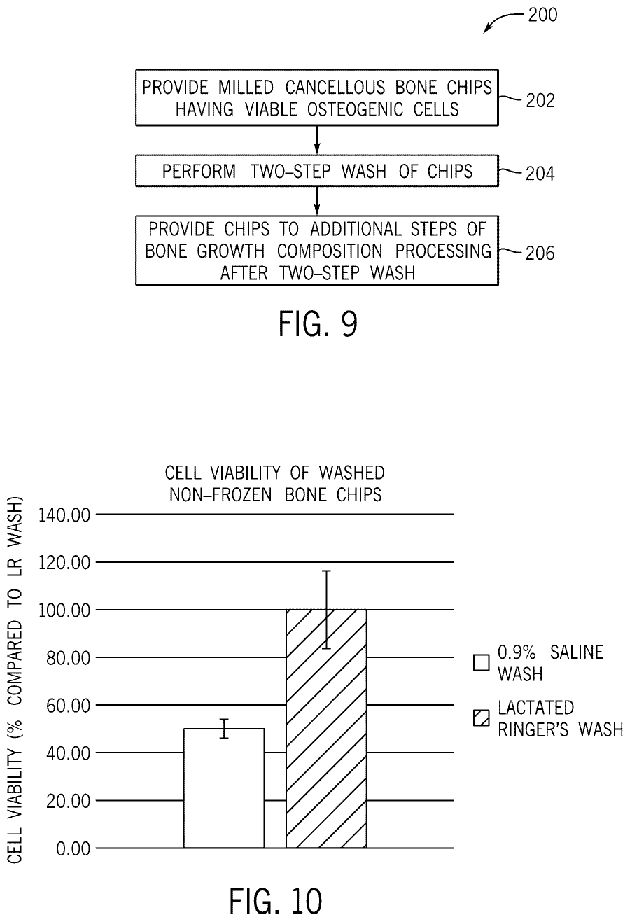

FIG. 9 is a method 200 of monitoring viability of osteogenic cells after a two-step washing process, as discussed with respect to FIGS. 4-5 and with reference to FIGS. 1-3. At step 202, milled cancellous bone chips are provided. The chips are washed using a two-step wash (see FIGS. 4-5) at step 204 as provided herein, and the washed chips are provided to additional steps of the bone growth composition processing at step 206.

FIGS. 10-11 show example cell viability data for different washing conditions. FIG. 10 is a comparison of cell viability, as assessed by PrestoBlue, of washed non-frozen tissue using saline to washed non-frozen tissue using Lactated Ringer's from a single donor. Lactated Ringer's as a wash solution demonstrated improved viability relative to saline. The cell viability is shown as a percentage comparison to Lactated Ringer's of 0.9% saline for washed, non-frozen chips.

FIG. 11 is a comparison of cell viability, as assessed by PrestoBlue, of different washing procedures assessed for a single donor. A single-step Lactated Ringer's wash by stirring or a single-step MicroAire lavage with Lactated Ringer's, did not perform as well as a two-step water (CPW denoting a clean room water source or filtered water) and Lactated Ringer's wash. The cell viability is shown as a cell viability percentage compared to a two-step water and Lactated Ringer's wash for a single-step Lactated Ringer's wash or single-step MicroAire lavage with Lactated Ringer's in which the two-step wash showed improved viability.

The disclosed techniques may use a glycerol cryosolution that can be used with direct freezing in certain embodiments. That is, packaged and sealed cellular bone matrix products may be placed directly in a conventional freezer, and do not necessarily require a controlled freezing process to maintain sufficient cell viability. In addition, in certain embodiments, the cellular bone matrix products as provided herein may be used without decanting liquid cryosolution from the thawed product and/or without rinsing or centrifugation. In certain embodiments, the product is thawed to 37° C. in a solution bath, and the packaged and sealed cellular bone matrix products can remain in the cryosolution at the thawing conditions for at least 2 hours or 4-6 hours after thawing without significant cell viability impacts.

In an embodiment, the frozen bone growth composition is in a solid state and transitions to a partially liquid state after thawing. That is, certain portions of the bone growth composition may remain solid while certain portions may be liquid. In an embodiment, the bone growth composition, after thawing, may have a flowable, slurry, or putty-like consistency. The term “flowable” may refer to compositions whose consistencies range from those which can be described as shape-sustaining but readily deformable, e.g., those which behave like putty, to those which are runny. Specific forms of flowable bone powder compositions include cakes, pastes, creams and fillers. Reference is made to U.S. Pat. No. 5,290,558, herein incorporated by reference in its entirety, for discussion of flowable materials. The bone growth composition may be a gel, putty, paste, cake, or solid in embodiments. The disclosed bone growth composition provides a product with sufficient cohesion for efficient manipulation of the product intraoperatively and, in embodiments, improved mixing with other materials (autograft and/or bone marrow aspirate, other bone graft materials, etc.), and improved bone defect filling and shaping to optimize bone defect repair.

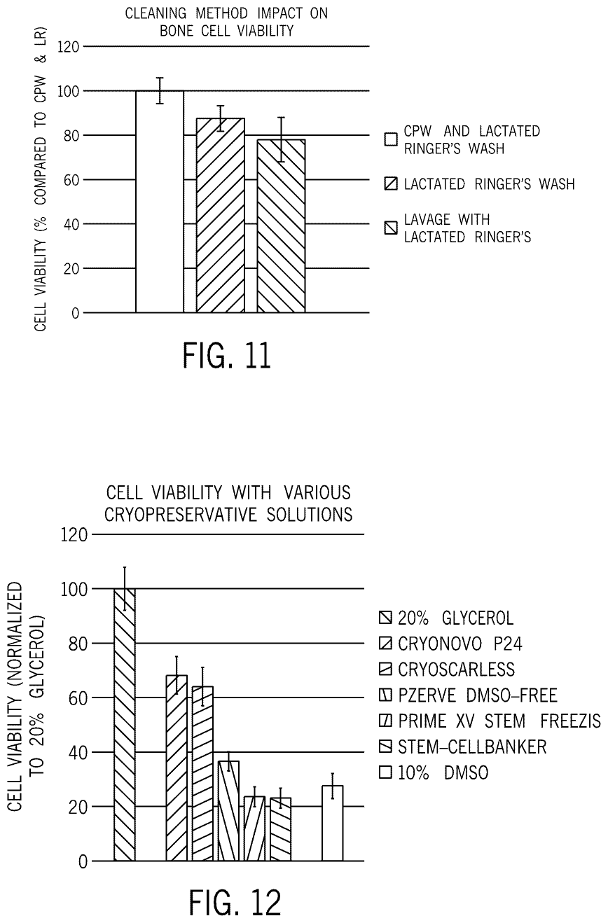

FIG. 12 shows a comparison of cell viability for a glycerol cryosolution in Lactated Ringer's (e.g., 80% Lactated Ringer's) relative to certain commercial cryosolutions demonstrating that 20% glycerol by volume outperforms multiple commercial cryosolutions under the same conditions. The cell viability is shown as a cell viability comparison normalized to the 20% glycerol by volume results for various other cryosolutions.

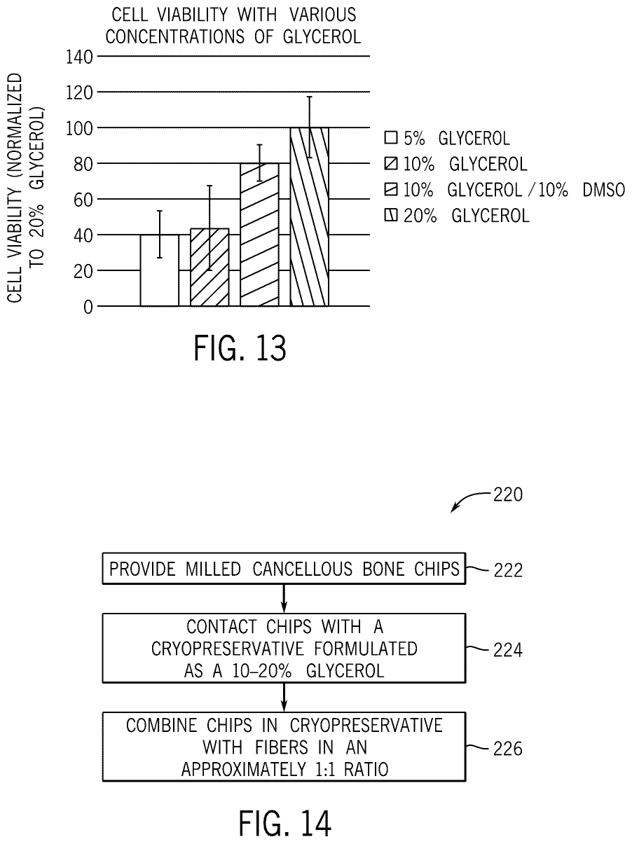

FIG. 13 shows cell viability for various glycerol concentrations in Lactated Ringer's. The cell viability is shown as a cell viability comparison for various glycerol concentrations in Lactated Ringer's normalized to 20% glycerol by volume.

FIG. 14 is a method 220 of combining chips and fibers during cellular bone matrix production and with reference to FIGS. 1-5. At step 222, milled cancellous bone chips are provided. At step 224, chips are contacted with a cryosolution or cryosolution as provided herein. In certain embodiments, the cryosolution contact occurs subsequent to the antimicrobial and/or antifungal contact and rinse. In certain embodiments, the rinse is Lactated Ringer's, and the rinse solution is removed to facilitate the cryosolution soak in the rinse solution with 10-20% glycerol by volume. In other embodiments, the glycerol may be directly added to the rinse solution. The soak may be conducted at 22-30° C. in an embodiment.

After the appropriate amount of time has elapsed (e.g., a minimum of 60 minutes to a maximum of 180 minutes in an embodiment), the excess cryosolution of the chips can be decanted, leaving enough solution behind to keep the combined cellular bone matrix product wetted. At step 226, the chips and fibers are combined in an approximate 1:1 ratio. By way of example, chips and fibers can be aliquoted using a same-sized volume (e.g., cubic centimeters) measure. The combined material can be filled into various pouches according to desired size and sealed. The chips and fibers, together with the cryosolution, may be measured according to total volume, such as cubic centimeters. In an embodiment, the liquid of the cryosolution may be measured in mL. For example, a bone growth composition may be aliquoted using 1-15 cubic centimeters of fibers and 1-15 cubic centimeters of chips. The chips and fibers may carry liquid that, in the packaging, forms the cryosolution. This solution may be present in liquid volume amounts of 1-10 mL (e.g., 1-50 cc), in an embodiment. In an embodiment, the excess (e.g., free or decantable) total liquid amount in the packaging is 0-2 mL and a total amount of the fibers or chips is 1-10 cubic centimeters or 6-8 cubic centimeters.

It should be understood that the disclosed compositions are wetted and, therefore, the cryosolution that is present may be associated with, absorbed by, or otherwise coupled together with the bone tissue. Thus, more liquid may be present than is decantable within the package. In contrast to less viable compositions, the disclosed wetted bone growth compositions are exposed to associated cryosolution, even after thawing, and nonetheless maintain significant viability.

In an embodiment, any remaining solution in the combined chips and fibers composition has a cryosolution, e.g., glycerol, present in a range of 1-25%. In certain embodiments, the fibers, stored in Lactated Ringer's, are drained prior to combination. Thus, little or no Lactated Ringer's from the stored fibers may be carried over into the combined composition, and a majority of the solution present is the cryosolution. Accordingly, chips in a cryosolution having 20% glycerol may yield a cellular bone matrix composition having 1-20% glycerol in an embodiment. In certain embodiments, the Lactated Ringer's solution is present in about equal volume as the cryosolution. By way of example, in a 1:1 ratio combination, the existing cryosolution is halved in percentage after combination. Accordingly, a cryosolution having 20% glycerol has about 10% glycerol after combination in such an embodiment. In certain embodiments, the fibers are retained in Lactated Ringer's prior to combination while the chips have been drained or partially drained. Accordingly, a cryosolution having 20% glycerol has less than 10% glycerol after combination in such an embodiment.

The disclosed cellular bone matrix production techniques incorporate a cryosolution contact step to maintain cell viability at cryogenic temperatures. However, cryosolution agents, while protective at cryogenic temperatures, may be cytotoxic at temperatures higher than the cryogenic temperatures. Thus, cryosolutions have potential to reduce cell viability both during the freezing process as well as during the thawing process. In addition, the time spent in contact with the cryosolution after thawing is dependent on the end user preparation conditions and preferences. Certain techniques may involve a decanting and/or centrifugation step post-thawing to remove the cryosolution present in the thawed sample. However, these steps may also be associated with loss of viable cells to handling. In addition, certain techniques may also involve a controlled freezing process that involves a specialized freezing container with a linear temperature decrease per minute to reduce cell loss as a result of cryosolution contact during the freezing process.

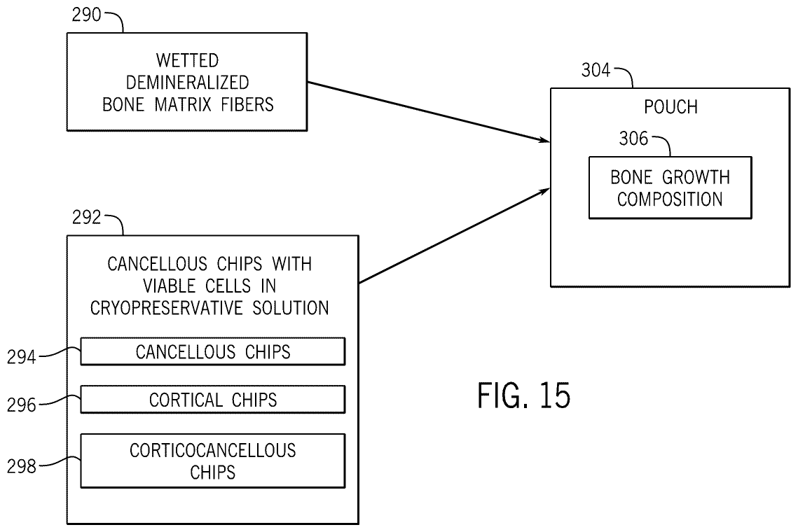

FIG. 15 shows an example bone growth composition product in which wetted demineralized bone matrix fibers 290 are combined with a cancellous chip mixture in cryosolution 292 having viable cells that are in cryosolution. As discussed herein, the cancellous chips in cryosolution 292 may include a mix of cancellous chips 294, cortical chips 296, and corticocancellous chips 298. The chips 292 may be predominantly cancellous chips 294 (e.g., 50-90% or greater) with the remainder being cortical chips 296 and/or corticocancellous chips 298.

The fibers 290 and cancellous chips in cryosolution 292 are packaged in a container, shown as a pouch 304, to form a cryo-ready bone growth composition 306. The pouch 304 is sealed, and the bone growth composition 306 is frozen, stored, and/or transported for use. Thus, the bone growth composition product may, in embodiments, include both the packaging, such as the pouch 304, and the bone growth composition 306.

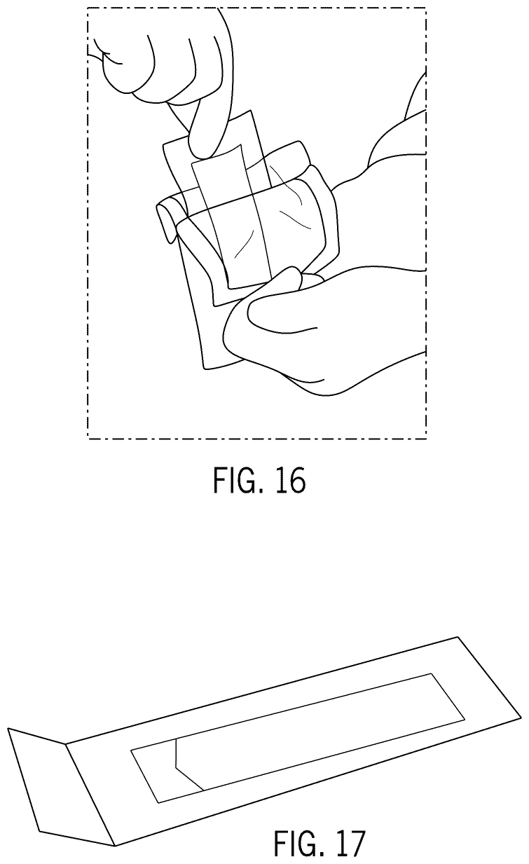

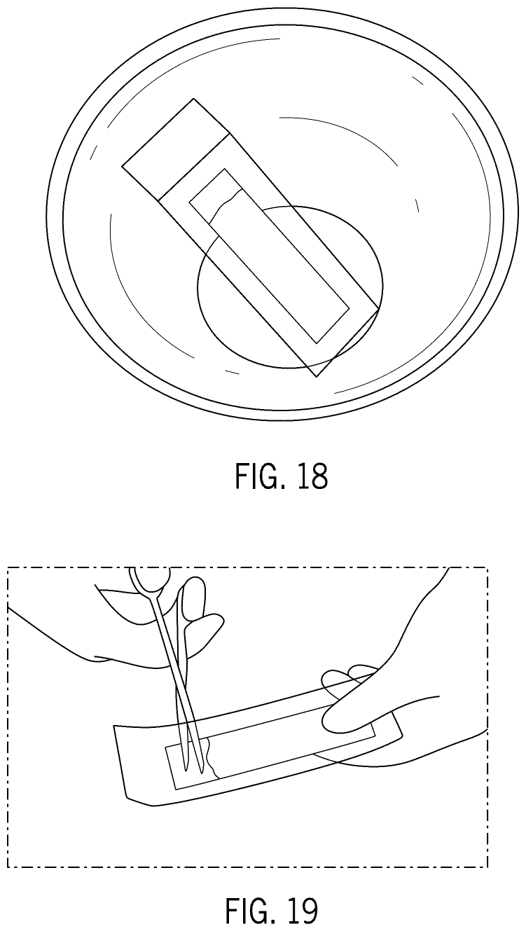

FIG. 16 is an image of an example packaging including a sealed interior pouch with bone growth composition being aseptically removed from a protective outer packaging. FIG. 17 shows the sealed interior pouch with bone growth composition. FIG. 18 shows thawing of bone growth composition in the sealed interior pouch. FIG. 19 shows an end user opening the interior pouch after thawing to access the bone growth composition.

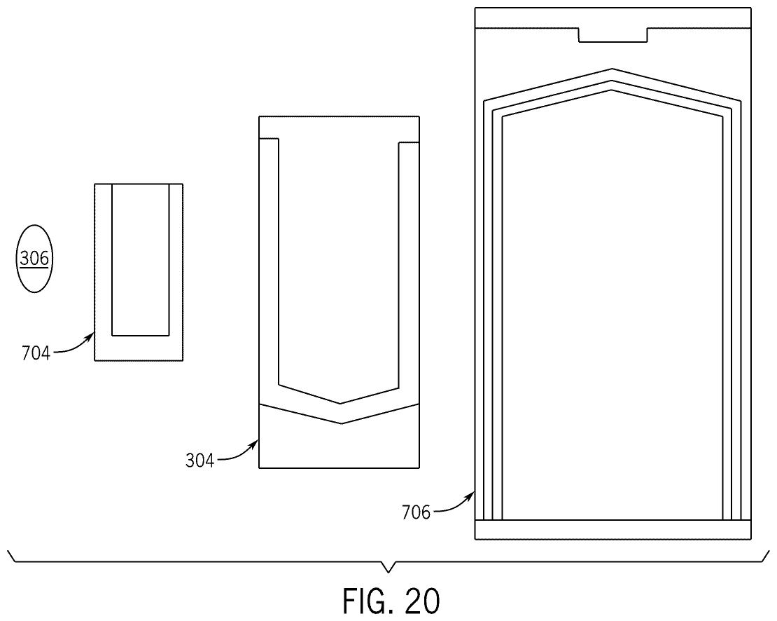

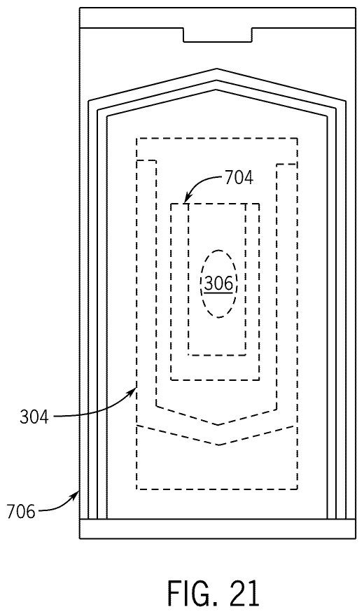

In certain embodiments, and as illustrated in FIGS. 20 and 21, the bone growth composition 306 may be “triple-pouched”, with the pouch 304 (e.g., main or intermediary pouch) containing a sterile sealed inner pouch 704 in which the bone growth composition 306 is contained. An outer or protective pouch 706 may in turn contain the pouch 304. In certain embodiments the pouch 304 and/or the outer or protective pouch 706 may be ClearFoil pouches or other suitable composition. In one embodiment the inner pouch 704 may be a nylon pouch, but in practice the inner pouch 704 may be fabricated using any medically suitable polymer or composition. In one such example, using suitable sterile techniques, the pouch 304 may be opened (e.g., peeled open using unsealed flaps at an end, cut open at a visually marked indicator, torn open along a perforation, and so forth) and the sterile inner pouch 704 containing the bone growth composition 306 may be aseptically accessed and transferred to a sterile field. By way of example, FIG. 20 depicts the three pouches of such an embodiment separate and relative to one another while FIG. 21 depicts the three pouches of such an embodiment in a practical implementation for storage and transport, with the pouch 304 placed within the outer pouch 706, the inner pouch 704 placed within the pouch 304, and the bone growth composition 306 placed within the inner pouch 704.

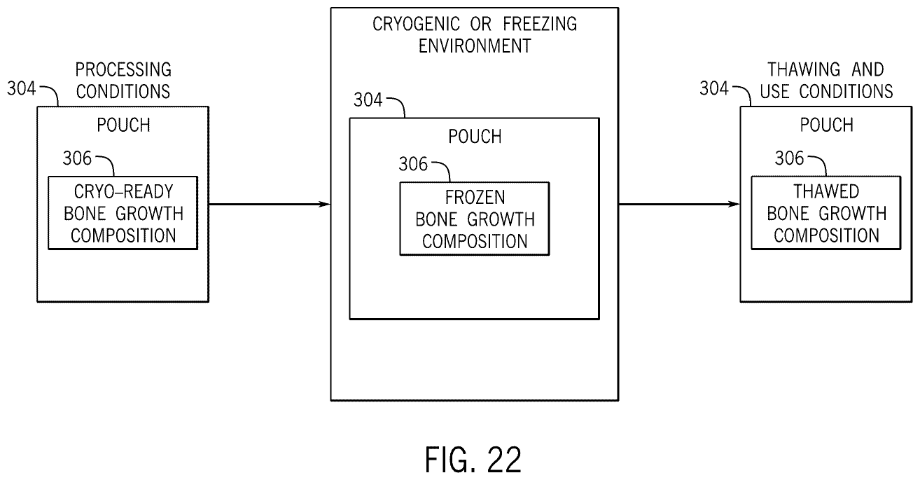

FIG. 22 shows the bone growth composition packaging in different conditions. At the end of processing of the donor tissue, as shown in FIG. 15, the fibers 290 and chips 292 (e.g., chip mixture) are combined to form the cryo-ready bone growth composition 306 that is packaged and sealed in the pouch 304. The processing conditions may be room temperature conditions, e.g., 20-25° C. After packaging, the pouch is placed in a freezing environment, e.g., a cryogenic environment, and the bone-growth composition 306 in the pouch transitions from a room temperature or nonfrozen state to a frozen bone growth composition 306. When the bone growth composition 306 is ready for use in a patient, the pouch 304 is removed from the freezing environment and thawed, and the frozen bone growth composition 306 transitions to a thawed bone growth composition 306.

In an embodiment, the number of viable cells of the bone growth composition 306 present at the time of packaging is reduced by less than 30% or less than 50% after thawing.

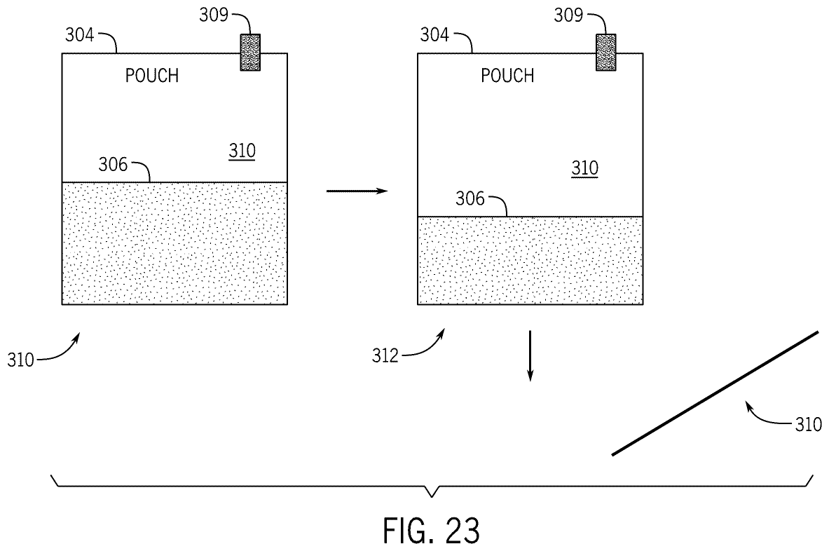

FIG. 23 shows a bone growth composition packaging system in which the pouch 304 includes features to permit decanting of liquid via a port 309 that, when opened, breaks the pouch seal. Additionally or alternatively, the pouch 304 may include a release liner 310 that can be peeled away or removed to reveal a perforated wall 312 including perforations that extend from an exterior of the pouch 304 through to an interior space 310 to permit draining of cryosolution liquid via the perforations. The perforations may be sized to retain chips and fibers in the pouch 304 while permitting liquid drainage.

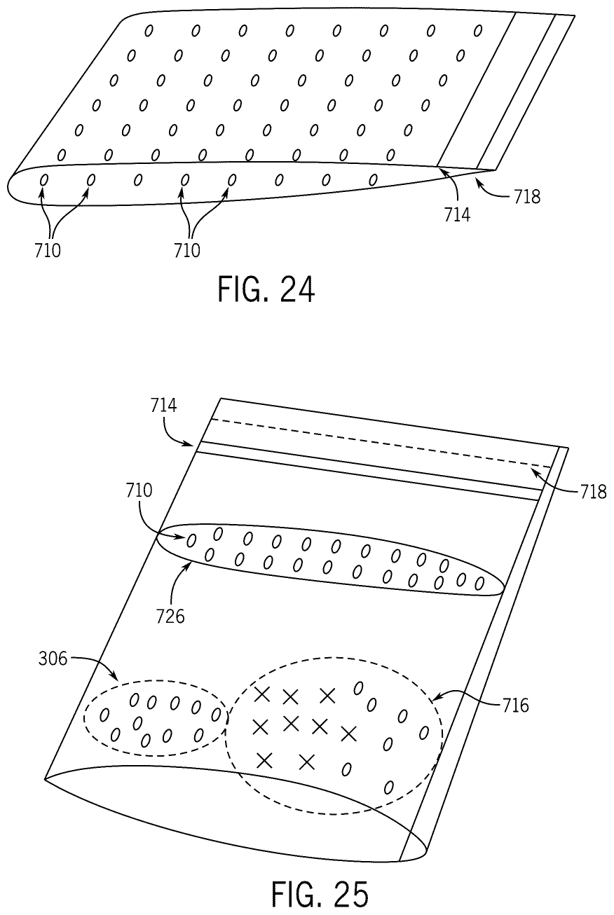

By way of further example, FIGS. 24 and 25 depict an example of a packaging and transportation approach incorporating integrated drainage solution. By way of example, and turning to FIG. 24, a pouch or bag (e.g., pouch 304 or inner pouch 704 is depicted that incorporates a plurality of perforations or drain features 710 through which cryosolution may be drained without loss of bone growth composition 306 (e.g., microsize). In such an embodiment the drainage features may have a uniform, staggered, or random placement pattern and may positioned on one or more sidewalls of the pouch. Such a pouch or package having integrated drainage features would be sealed or otherwise stored in a larger pouch or bag (e.g., pouch 304 or outer pouch 706) through which the cryosolution may flow during storage and into which the cryosolution may during preparation. By way of example, in such an embodiment a user may open the outer pouch or bag and, in the process of lifting or removing the inner pouch or bag having the bone growth composition, the cryosolution may be drained from the inner pouch simply by the act of lifting or partially removing the inner, perforated pouch from the outer pouch. As discussed in other embodiments, such a pouch that incorporates integrated drainage features may include an integrated sealing feature 714 (e.g., a user-activated complementary sealing feature, such as an interlocking groove and ridge seal mechanism) and/or a built-in release mechanism to access the bone growth composition, such as a perforated tear-off region 718. In practice, the two pouches may be integrated as nested or attached pouches having integrated drainage features (e.g., as a bag-in-a-bag solution). In such an integrated approach, the two pouches may be in fluid communication with respect to the cryosolution when the drainage features are exposed.

Turning to FIG. 25, a partially transparent view of one embodiment is depicted. In the depicted example, the pouch incorporating the integrated drainage features may incorporate such features (e.g., perforations) in a diaphragm region 726 (e.g., an inner wall or inner membrane). such perforations may be sealed prior to use for draining, such as by a removeable seal or layer that may be chemically adhered and/or structurally attached until removed, at which point the drainage features may be exposed and used. In this manner, pouch may be fluid tight up until a seal is removed to expose the drainage features 710, at which point the cryosolution may be drained prior to use. In the depicted example, the pouch may include an integrated sealing feature 714 and/or a built-in release mechanism, such as a perforated tear-off region 718, as discussed herein. In such an example, a user may open the pouch may tearing or opening along the tear-off region 718, may insert autograft material 716 into the pouch along with the bone growth composition 306, and may reseal the pouch using integrated sealing feature 714. Once the autograft material 716 is added and the pouch is resealed, a user may knead or manipulate the sealed pouch so as to mix the bone growth composition 306 and the autograft material 716. Once mixed, the user may unseal or expose the integrated drainage features 710 and pour off the cryosolution.

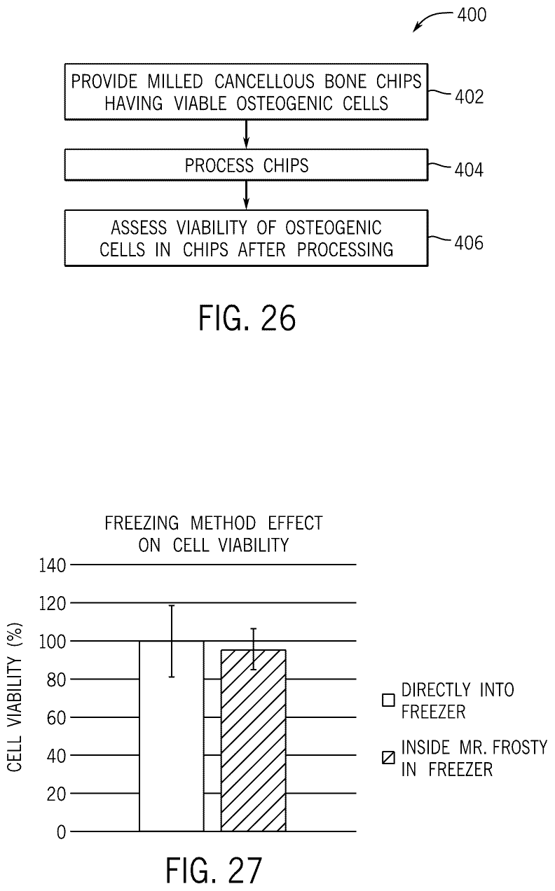

As provided herein, osteogenic may refer to the ability of a material to enhance or accelerate the growth of new bone tissue by one or more mechanisms such as osteogenesis, osteoconduction, and/or osteoinduction. FIG. 26 is a method 400 of monitoring viability of osteogenic cells after chip processing. At step 402, milled cancellous bone chips are provided. The chips are processed at step 404 as provided herein, and the viability of the osteogenic cells is assessed at step 406. In the illustrated embodiment, the viability is assessed after processing, e.g., after the chips are contacted with the cryosolution. Viability may additionally be assessed following contact with the antimicrobial and/or antifungal agent and/or after thawing.

In an embodiment, the viability is assessed using a modified PrestoBlue assay. Viable cells are able to convert resazurin (blue) to resorufin (pink). The absorbance indicative of pink color conversion can be compared against a standard curve based on known numbers of viable cells to estimate a viable cell count in a sample.

As provided herein, the cancellous chips may have an estimated viable cell concentration of at least 50,000 viable cells/cc.

Osteoinductivity, may refer to the quality of being able to recruit cells from the host that have the potential to stimulate new bone formation. Any material that can induce the formation of ectopic bone in the soft tissue of an animal is considered osteoinductive. For example, most osteoinductive materials induce bone formation in athymic rats when assayed according to the method of Edwards et al., “Osteoinduction of Human Demineralized Bone: Characterization in a Rat Model,” Clinical Orthopaedics & Rel. Res., 357:219-228, December 1998. In other instances, osteoinduction is considered to occur through cellular recruitment and induction of the recruited cells to an osteogenic phenotype. Osteoinductivity score refers to a score ranging from 0 to 4 as determined according to the method of Edwards et al. (1998) or an equivalent calibrated test. In the method of Edwards et al., a score of “O” represents no new bone formation; “1” represents 1%-25% of implant involved in new bone formation; “2” represents 26-50% of implant involved in new bone formation; “3” represents 51%-75% of implant involved in new bone formation; and “4” represents >75% of implant involved in new bone formation. In most instances, the score is assessed 28 days after implantation. However, the osteoinductivity score may be obtained at earlier time points such as 7, 14, or 21 days following implantation. Percentage of osteoinductivity refers to an osteoinductivity score at a given time point expressed as a percentage of activity, of a specified reference score.

As provided herein, the cellular bone matrix thawed from frozen may have an osteoinductivity score of at least 1 or at least 2 based on a 50:50 chip/fiber formulation. Results of osteoinductivity tests for cellular bone matrix products prepared according to the disclosed techniques are shown below in Table 1. The osteoinductivity tests were performed as in Edwards et al. and using a rat muscle pouch assay. Table 2 demonstrates that the osteoinductivity of the tissue was maintained during extended storage at −70° C.

TABLE 1
Osteoinductivity scores

| Description | OI Score |
|---|---|
| 50% chips/50% fibers | 1.83 ± 0.75 |
| (n = 8 lots) | |
| 100% fibers | 2.96 ± 0.62 |
| (n = 4 lots) | |

TABLE 2
Osteoinductivity is maintained following storage at −70 C. In
the testing conditions, samples were stored in the freezer at −70
C. for an extended amount of time (shown in table 2) and tested again
in the rats. The results confirm that the tissue retained its osteoinductivity.
On average, the osteoinductive score for the disclosed cellular
bone matrix compositions is higher than that of tested competitive
products. By way of example, OI scores were assessed for competitive
products as follow: OI for Osteocel Pro (n = 3 lots) was 0.9
± 0.1. For OsteoCel Plus (n = 5 lots), it was 0.2 ±
0.3. Map3 (no longer on market) was 0.25 ± 0.0 (n = 3 lots).

| Group | Description | Original Score | Re-test Score |
|---|---|---|---|
| 1 | 50% chips/50% fibers | 2.25 | 2.00 (after 1 year) |
| 2 | 100% fibers | 3.50 | 3.25 (after 1 year) |
| 3 | 100% fibers | 3.00 | 3.50 (after 6 months) |

The disclosed cellular bone matrix compositions are associated with relatively higher viable cell counts and longer shelf lives and/or longer time windows for use after thawing as a result of improved processing. In one example, disclosed cellular bone matrix products maintain viability for at least 2 hours or at least 4-6 hours after thawing and without separation from the cryosolution. In an embodiment, the total thaw time for a 15 cubic centimeter (as packaged) size is 6 minutes or less.

FIG. 27 is a percentage comparison between the direct and controlled freezing showing very similar viability results. As shown, the use of a controlled freezing device provided no cell viability benefit under the assessed conditions. Accordingly, in certain embodiments, the pouches or other containers may be placed directly in cryogenic conditions, such as a −70° C. freezer, without use of the controlled freezing device. A benefit of the disclosed production techniques for cellular bone matrix compositions is a less complex and more user-friendly freezing process.

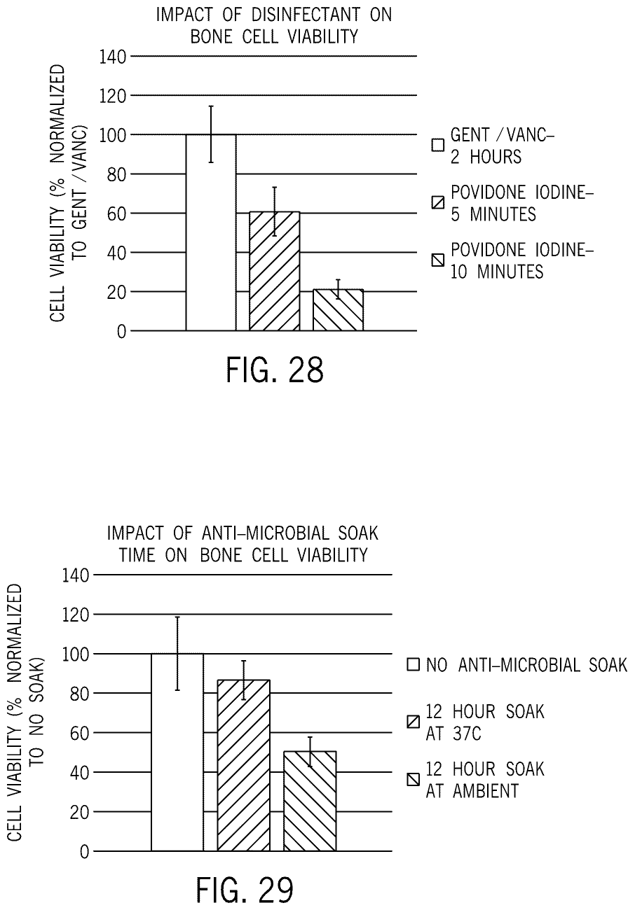

FIGS. 28-31 show results from different antimicrobial agent soaks and contact conditions. FIG. 28 shows results of different soak times and antimicrobial agents, such as a gentamicin and vancomycin or 1% povidone-iodine. The cell viability is shown as with results normalized to a 2-hour gentamicin and vancomycin soak. The presence of povidone iodine was associated with decreased cell viability.

FIG. 29 shows benefits of performing the antimicrobial contact step at temperatures above room temperature. A 12-hour antimicrobial soak at 37° C. demonstrated elevated results relative to room temperature soaks at various lengths. The cell viability is shown normalized to a no antimicrobial contact control.

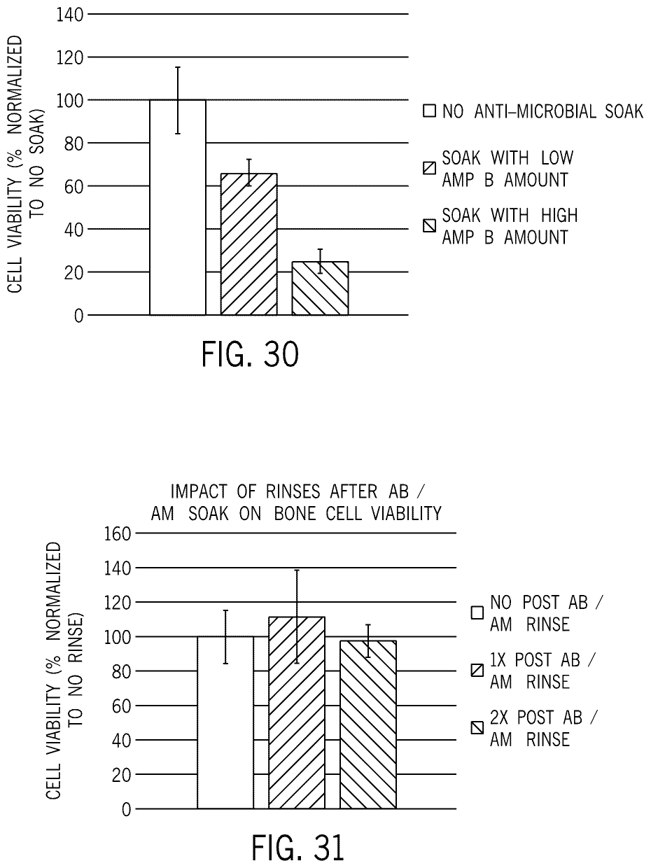

FIG. 30 shows results of different anti-microbial concentrations. A concentration-dependent decrease in bone cell viability was observed.

FIG. 31 shows effects on cell viability using different post-antimicrobial soak rinse conditions results relative to a no rinse control. Including at least one rinse step did not reduce cell viability.

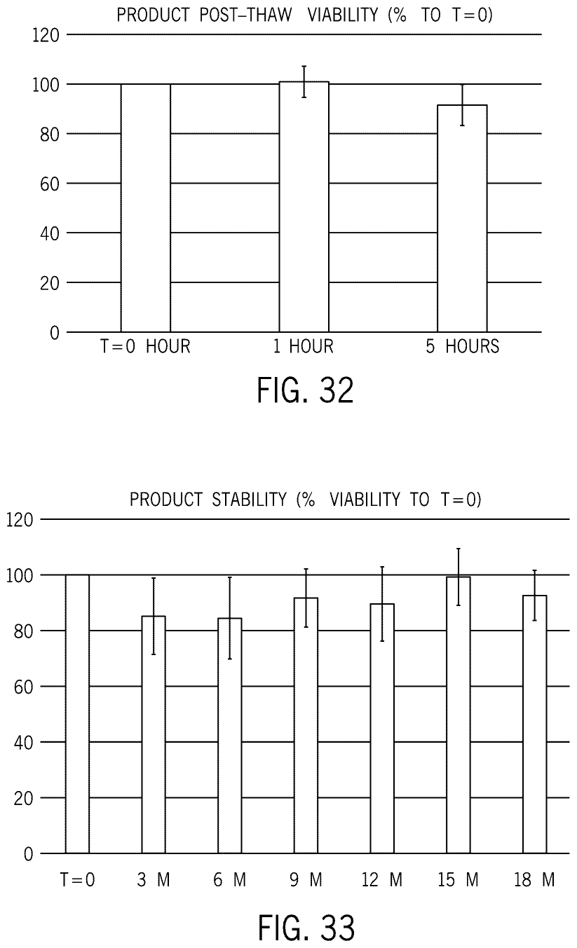

FIG. 32 shows post-thaw viability data for the bone growth composition of approximately, 100% at 1 hour and 90% at 5 hours normalized to the number of cells viable immediately after thawing (time zero or T=0). FIG. 33 shows post-thaw viability data for the bone growth composition of approximately 90% after 3, 6, 9, 12, 15, and 18 months of storage at −70° C. normalized to the number of cells when the tissue was first placed into the freezer at the end of processing (time zero or T=0).

The disclosed techniques provide improvements in maintaining the cells, tissues, or other biologically active components of a bone growth composition from harvesting through processing and until the composition is readied for transplantation into a recipient. During the interval between harvest and implantation, it is also desirable to monitor and control the environmental conditions, and storage parameters to maintain the integrity, viability, and biochemical activity of the harvested and processed biological material from which the bone growth composition is formed.

Thus, the disclosed bone growth compositions have improved viability and/or biologic activity. While it is contemplated that the parameters of storage temperature, atmospheric pressure, ambient environmental conditions, and such like will provided to users of the bone growth composition, the bone growth compositions have a wider tolerance of storage and handling conditions to reduce the effects of end user variability and preferences on the therapeutic effectiveness of the disclosed bone growth compositions. For example, not all end users have access to stringent storage conditions, such as a −70° C. freezer. However, the disclosed bone growth compositions maintain suitable activity in a conventional −20° C. freezer. The bone growth composition has been demonstrated to be viable (e.g., greater than 80% viability) after 5 days at −20° C. In particular, viability was observed to be 100% after 5 days at −20 C. and 96% after 15 days at −20 C. In addition, viability was observed to be 95% after 4 weeks at −40 C and 71% after 8 weeks at −40 C. With this in mind, because freezers may be subject to variability in operating conditions, the disclosed bone growth compositions are able to maintain sufficient viability through fluctuations in freezer operating conditions, such that intermittent changes of temperature within a tolerance band (e.g., −15° C. to −25° C.) will not significantly alter the therapeutic effectiveness of the disclosed bone growth compositions. As may be appreciated, although the present discussion primarily describes a polymeric pouch as a suitable vessel for storage and/or thawing of the bone growth composition, in practice any suitable vessel may be employed, including but not limited to jars, syringes, cannulas, and the like, which may in practice be formed from plastic or other biosafe materials that are safe and/or stable at low temperatures. Further, various structural features and/or markings may be provided to allow accessing the bone growth composition when it is ready to be used or applied. By way of example, such features may include, but are not limited to, tearable perforations provided on the pouch, separate layers of the pouch that may be peeled apart to separate a sealed inner portion of the pouch, a visually marked or nocked region of the pouch indicating where the pouch may be cut open, and so forth. In certain embodiments, as discussed elsewhere herein, two or more pouches (or comparable structures) may be provided, such as an inner pouch. By way of example, in one such embodiment an inner pouch may contain the bone growth composition while a main or outer pouch or pouches may be configured to allow addition (such as via a rubber gasket structure) of a thawing or temperature modulating medium, such as a thawing or warming fluid suitable for facilitating thawing of the bone growth composition at a rate suitable for preserving cell viability and minimizing time needed for thawing before use in a procedure.

The disclosed techniques are directed to bone growth compositions and methods. In certain embodiments, the bone growth compositions and methods may be cryogenically preserved. In an embodiment, cryogenic preservation, e.g., freezing, refers to exposing a population of cells, tissues, or organs to a cryogenic environment. A cryogenic environment may refer to temperatures below 0° C., below −20° C., below −70° C. In an embodiment, a cryogenic environment may be between about 0° C. to about −200° C.

A cryoprotectant or cryosolution may refer to agents or materials that prevents or reduces undesirable damage to a biological material caused by lowering the temperature of a biological material or any substance or material which enhances, strengthens or otherwise increases the ability of the biological material to withstand lowered temperatures. As disclosed herein, the bone growth compositions and associated cryopreservation methods may include the addition of one or more cryoprotectants or cryosolution compounds to permit freezing of the sample, and/or maintenance of the sample at temperatures generally below 0° C. Exemplary cryoprotectants and/or cryosolution compounds, as used in the context of the disclosed techniques may include, but are not limited to, ice-suppressing cryoprotectants (e.g., non-colligative agents such as Supercool X-1000™ and Supercool Z-1000™, 21^{st} Century Medicine, Rancho Cucamonga, Calif.) glycerol, dimethylsulfoxide (DMSO), ethylene glycol, propylene glycol, polyethylene oxide (PEO), acetamide, ethanol, methanol, butanediol, carbohydrates (including sugars such as glucose, fructose, dextrans, sucrose, lactose, and trehalose), polyvinyl alcohols, hydroxyethyl starch, serum albumin. With CBMs, cryopreservation preserves the osteogenic potential of allogeneic cells providing benefits for the bone grafting site.

In certain embodiments, steps that involve freezing and/or thawing of a tissue sample or cell population may be achieved by, e.g., bringing the temperature of a refrigerated tissue or cell sample down to a suitable sub-zero temperature, or alternatively, bringing the temperature of a sub-zero stored sample up to refrigerated (and, optionally, to either room or recipient body temperature immediately prior to implantation). Freezing may include direct freezing a room temperature composition. Such steps in the disclosed methods may employ submersion vessels or frozen storage means to prepare the frozen tissue or cell sample, while conventional means such as a heated water bath or such like device, submerging the packaged frozen sample into a sample of growth medium, biological buffer, or tissue/organ storage solution (e.g., pre-warmed to the desired temperature), may be employed to bring the temperature of a frozen tissue sample to the desired temperature required for transplanting the bone growth compositions into the body of a suitable recipient.

It should be understood that exposure to a cryogenic environment may result in the freezing of liquid portions of a bone growth composition. However, the resultant bone growth composition may include composite structures that do not change in state between freezing and thawing and/or that change state at different temperatures relative to water in the sample. Thus, in an embodiment, a bone growth composition as provided herein may be referred to as frozen or may freeze at temperatures that cause liquid portions of the composition to transition to a solid state.

As provided herein, antimicrobial includes, for example, antibiotics, antifungal, antiviral agents or the like. Antimicrobial agents to treat infection include by way of example and not limitation, antiseptic agents, antibacterial agents; polyene antifungals (e.g., amphotericin B), quinolones and in particular fluoroquinolones (e.g., norfloxacin, ciprofloxacin, lomefloxacin, ofloxacin, etc.), aminoglycosides (e.g., gentamicin, tobramycin, etc.), glycopeptides (e.g., vancomycin, etc.), lincosamides (e.g., clindamycin), cephalosporins (e.g., first, second, third generation) and related beta-lactams, macrolides (e.g., azithromycin, erythromycin, etc.), nitroimidazoles (e.g., metronidazole), penicillins, polymyxins, tetracyclines, or combinations thereof. Some exemplary antimicrobial agents include, by way of illustration and not limitation, acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chloroxylenol; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; chlorhexidine, cloxyquin; colistimethate sodium; colistin sulfate; coumermycin; coumermycin sodium; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; tromethamine; fumoxicillin; furazolium chloride; furazolium tartrate; fusidate sodium; fusidic acid; ganciclovir and ganciclovir sodium; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine sodium; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodium; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin and oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillins such as penicillin g benzathine, penicillin g potassium, penicillin g procaine, penicillin g sodium, penicillin v, penicillin v benzathine, penicillin v hydrabamine, and penicillin v potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin b sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin; zorbamycin; or combinations thereof.

Certain embodiments of the disclosure are discussed in the context of cellular bone matrices or bone matrices. However, it should be understood that one or more compositions or processing steps may be used for other bone growth compositions. For example, the disclosed process steps may be used alone or in combination with other type of bone growth processing that includes one or both of chips or demineralized fibers.

The disclosed bone growth compositions may include a bioactive or pharmaceutical agent, which may be disposed in, packaged with, coated on or combined with the bone growth composition. The term “bioactive agent” as used herein is generally meant to refer to any substance that alters the physiology of a patient. The term “bioactive agent” may be used interchangeably herein with the terms “therapeutic agent,” “therapeutically effective amount,” and “active pharmaceutical ingredient”, “API” or “drug”.

Bioactive agent or bioactive compound is used herein to refer to a compound or entity that alters, inhibits, activates, or otherwise affects biological or chemical events. For example, bioactive agents may include, but are not limited to, osteogenic or chondrogenic proteins or peptides, anti-AIDS substances, anti-cancer substances, antibiotics, immunosuppressants, anti-viral substances, enzyme inhibitors, hormones, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and antiadhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, angiogenic factors, anti-secretory factors, anticoagulants and/or antithrombotic agents, local anesthetics, prostaglandins, anti-depressants, anti-emetics, and imaging agents. In certain embodiments, the bioactive agent is a drug. Bioactive agents further include RNAs, such as siRNA, and osteoclast stimulating factors. In some embodiments, the bioactive agent may be a factor that stops, removes, or reduces the activity of bone growth inhibitors. In some embodiments, the bioactive agent is a growth factor, cytokine, extracellular matrix molecule or a fragment or derivative thereof, for example, a cell attachment sequence such as RGD.

In some embodiments, the pharmaceutical agent may include one or a plurality of therapeutic agents and/or pharmacological agents for release, including sustained release, to treat, for example, pain, inflammation, degeneration, or infection. The agent may be an analgesic agent including but are not limited to acetaminophen, a local anesthetic, such as for example, lidocaine, bupivicaine, ropivacaine, opioid analgesics such as buprenorphine, butorphanol, dextromoramide, dezocine, dextropropoxyphene, diamorphine, fentanyl, alfentanil, sufentanil, hydrocodone, hydromorphone, ketobemidone, levomethadyl, levorphanol, mepiridine, methadone, morphine, nalbuphine, opium, oxycodone, papaveretum, pentazocine, pethidine, phenoperidine, piritramide, dextropropoxyphene, remifentanil, sufentanil, tilidine, tramadol, codeine, dihydrocodeine, meptazinol, dezocine, eptazocine, flupirtine or a combination thereof.

The agent may be an anti-inflammatory agent including, but are not limited to, a statin, sulindac, sulfasalazine, naroxyn, diclofenac, indomethacin, ibuprofen, flurbiprofen, ketoprofen, aclofenac, aloxiprin, aproxen, aspirin, diflunisal, fenoprofen, mefenamic acid, naproxen, phenylbutazone, piroxicam, meloxicam, salicylamide, salicylic acid, desoxysulindac, tenoxicam, ketoralac, flufenisal, salsalate, triethanolamine salicylate, aminopyrine, antipyrine, oxyphenbutazone, apazone, cintazone, flufenamic acid, clonixeril, clonixin, meclofenamic acid, flunixin, colchicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, triflumidate, fenamates (mefenamic acid, meclofenamic acid), nabumetone, celecoxib, etodolac, nimesulide, apazone, gold, tepoxalin; dithiocarbamate, or a combination thereof. Anti-inflammatory agents also include other compounds such as steroids, such as for example, fluocinolone, cortisol, cortisone, hydrocortisone, fludrocortisone, prednisone, prednisolone, methylprednisolone, triamcinolone, betamethasone, dexamethasone, beclomethasone, fluticasone interleukin-1 receptor antagonists, thalidomide (a TNF-α release inhibitor), thalidomide analogues (which reduce TNF-α production by macrophages), bone morphogenetic protein (BMP) type 2 or BMP-4 (inhibitors of caspase 8, a TNF-α activator), quinapril (an inhibitor of angiotensin II, which upregulates TNF-α), interferons such as IL-11 (which modulate TNF-α receptor expression), and aurin-tricarboxylic acid (which inhibits TNF-α), guanidinoethyldisulfide, or a combination thereof. Exemplary anti-inflammatory agents include, for example, naproxen; diclofenac; celecoxib; sulindac; diflunisal; piroxicam; indomethacin; etodolac; meloxicam; ibuprofen; ketoprofen; r-flurbiprofen; mefenamic; nabumetone; tolmetin, and sodium salts of each of the foregoing; ketorolac bromethamine; ketorolac tromethamine; ketorolac acid; choline magnesium trisalicylate; rofecoxib; valdecoxib; lumiracoxib; etoricoxib; aspirin; salicylic acid and its sodium salt; salicylate esters of alpha, beta, gamma-tocopherols and tocotrienols (and all their d, 1, and racemic isomers); methyl, ethyl, propyl, isopropyl, n-butyl, sec-butyl, t-butyl, esters of acetylsalicylic acid; tenoxicam; aceclofenac; nimesulide; nepafenac; amfenac; bromfenac; flufenamate; phenylbutazone, or a combination thereof.

An anti-inflammatory agent can be a steroid. Exemplary steroids include, for example, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, dexamethasone 21-acetate, dexamethasone 21-phosphate di-Na salt, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide or a combination thereof.

Anti-inflammatory agents also include those with anti-inflammatory properties, such as, for example, amitriptyline, carbamazepine, gabapentin, pregabalin, clonidine, or a combination thereof.

With the preceding in mind, further examples are provided with respect to bone growth composition application techniques, including devices suitable for use in applying a bone growth composition, such as a thawed or flowable bone growth composition as discussed herein. In certain embodiments a specialized or special purpose device may be employed suitable for receiving the bone growth composition directly or a deformable container (e.g., packet, pouch, and so forth) of the thawed or flowable bone growth composition. Such an application device 500 may be configured to apply direct or indirect pressure, such as to a portion of such a container, so as to cause the flowable bone growth composition to be extruded from an extrusion location (e.g., application or applicator tip) of the application device 500.

As discussed herein, all or portions of an application device 500 for dispensing bone growth composition can be fabricated from biologically acceptable materials suitable for medical applications, including metals, synthetic polymers, ceramics and/or their composites. For example, the components used individually or collectively in the fabrication of an application device 500, embodiments of which are discussed below, can be fabricated from materials such as stainless steel alloys, commercially pure titanium, titanium alloys, Grade 5 titanium, super-elastic titanium alloys, cobalt-chrome alloys, superelastic metallic alloys (e.g., Nitinol, super elasto-plastic metals, such as GUM METAL®), ceramics and composites thereof such as calcium phosphate (e.g., SKELITE™), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), calcium sulfate, or other resorbable polymers such as polylactide, polyglycolide, polytyrosine carbonate, polycaroplaetohe and their combinations.

Various components of an application device 500 may have material composites, including the above materials, to achieve various desired characteristics such as strength, rigidity, elasticity, compliance, biomechanical performance, durability and radiolucency or imaging preference. The components of an application device 500, individually or collectively, may also be fabricated from a heterogeneous material such as a combination of two or more of the above-described materials. The components of an application device 500, examples of which are illustratively discussed below, may be monolithically formed, integrally connected or include fastening elements and/or instruments, as described herein.

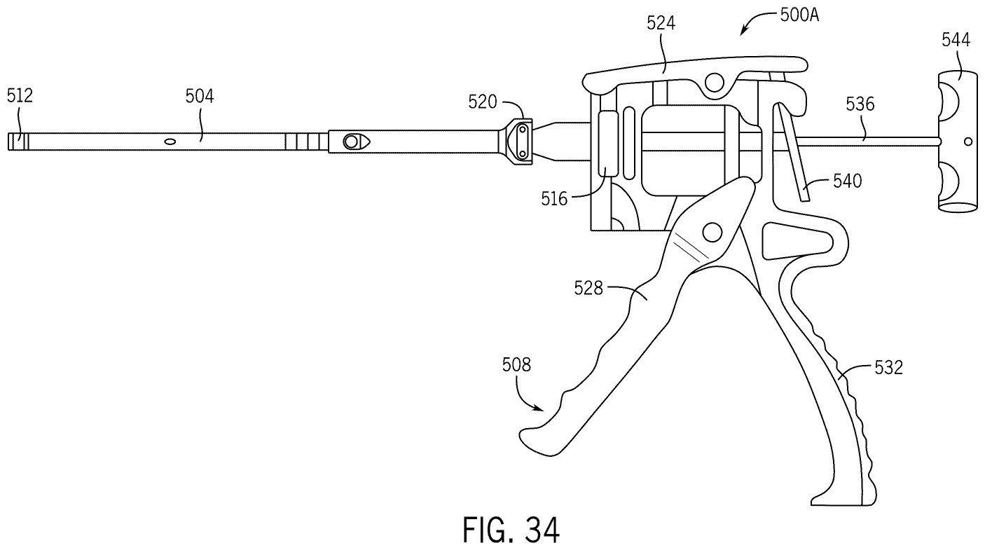

With the preceding in mind, and by way of example, in certain embodiments a bone growth composition delivery or application device may take the form of a handheld tool that may be actuated by hand and which dispenses bone growth composition, in response to such actuation. Such a tool may include a pressure source, with the extent of pressure being regulated by the manual actuation, such that the bone growth composition is dispensed at a known and/or adjustable rate and at a known location. With this in mind, FIG. 34 depicts an example bone growth composition application device in the form of a handheld tool, here depicted as what may be characterized as a bone-grafting injection gun. In particular, FIG. 34 shows an example bone growth composition application device 500A according to an aspect of the disclosure. In the depicted example, the application device 500A includes a delivery tube 504 and an actuation mechanism 508. The actuation mechanism 508 is configured to apply a force to bone growth composition material disposed in the tube 504 (either loose within the tube 504 or disposed within an insertable cartridge or canister that dispenses bone growth composition in response to pressure) causing the thawed and flowable bone growth composition to be dispensed through an open distal end 512 of the tube 504. The depicted embodiment of an actuation mechanism 508 includes a receiving mechanism 516 configured to receive and lock (or otherwise secure) the proximal end 520 (which may incorporate a loading funnel structure 772 as discussed herein) of the tube 504, so that the proximal end 520 of the tube 504 is adjacent to or abuts the actuation mechanism 508, allowing the actuation mechanism 508 to apply the force to the packaged or unpackaged bone growth composition. In the example device 500A of FIG. 34, the actuation mechanism 508 includes a frame 524 or similar structure which supports other components of the actuation mechanism 508, including components through which a user may actuate or otherwise manipulate the device 500A. The example device 500A includes a first handle 528 and a second handle 532. The example actuation mechanism 508 of FIG. 34 is configured to apply pressure (i.e., force) to the bone growth composition when the first handle 528 and second handle 532 are squeezed together. In some examples, the actuation mechanism 508 is mechanically coupled to a rod 536 and is configured to cause the rod 536 to advance toward the distal end 512 of the tube 504 when the actuation mechanism 508 is actuated. In some examples, a force is transmitted through the rod 536 to the bone growth composition packaging or to the composition itself in the absence of packaging. For example, each time a user squeezes the handles (528, 532) together, the actuation mechanism 508 causes the rod 536 to advance by a variable or fixed distance, where the rod 536 is either directly or indirectly in contact with the bone growth composition. In some embodiments the actuation mechanism 508 may include a ratchet mechanism so that as the handles are alternately squeezed and released, the rod 536 advances toward the distal end 512 of the tube 504 in a step-wise manner. Other actuation mechanisms are also within the scope of this disclosure.

The actuation mechanism 508 of FIG. 34 may also include a release mechanism 540 configured to release the force applied to the bone growth composition when the release mechanism 540 is pressed or otherwise manipulated. The force applied to the bone growth composition may be reduced by pulling on a handle 544 disposed at the end of the rod 536.

As discussed herein, the open distal end 512 of the tube 504 may be disposed at a target site, such as a vertebra or other bone of a subject undergoing a procedure. By squeezing the handles (528, 532) of the device 500A together, a user, such as a surgeon or other medical professional, may deliver a quantity of bone growth composition to the target site. In this manner, the application device 500A may accurately dispense a volume of bone growth composition

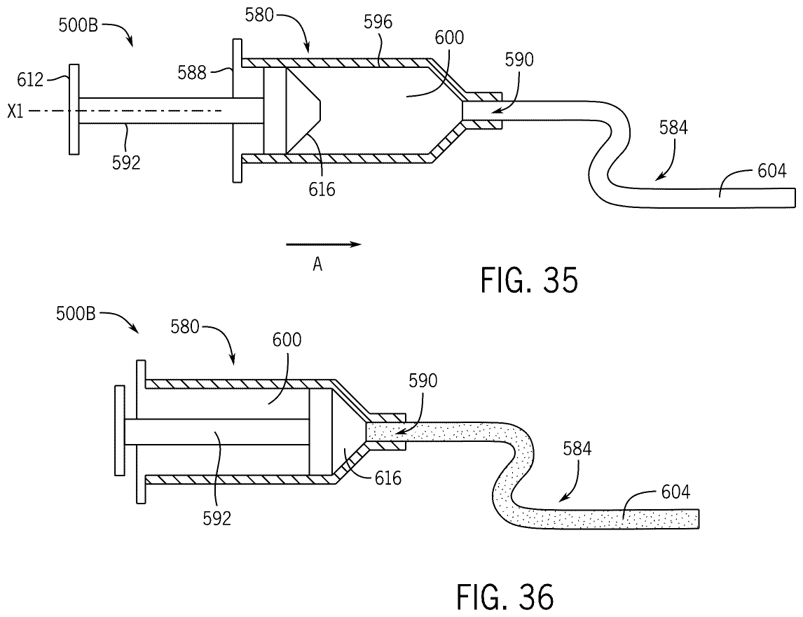

Turning to FIGS. 35 and 36, an alternative embodiment of an application device 500B for delivering bone growth composition to an anatomic site is depicted. The components of application device 500B can be employed, for example, for delivery and introduction of a bone growth composition as discussed herein. In some embodiments, application device 500B may be employed with certain medical procedures in which delivery of a bone growth composition is involved.

In the depicted example, application device 500B, which may be characterized as a syringe, comprises a plurality of elements (e.g., tubular elements) such as, for example, a syringe barrel 580 and tubing 584 that define a passageway. A volume of a thawed and/or flowable bone growth composition may be provided within the syringe barrel 580 and/or tubing 584 to facilitate application of the bone growth composition, via the tubing 584, to a selected anatomic site. Though depicted as flexible in FIGS. 35 and 36, the tubing 584 may in other embodiments be implemented as a rigid or fixed tube or applicator tip, such as a straight or angled rigid hollow needle that is inflexible.

In the depicted example, barrel 580 includes a proximal end (e.g., a “user-facing” end) and a distal end (e.g., a “patient-facing” end). An opening 588 defined at the proximal end is configured to accommodate an actuator, such as, for example, a plunger 592, as described herein. The distal end includes an opening 590 and is configured for connection with tubing 584 (e.g., a flexible applicator tubing, rigid hollow needle, and so forth), as described herein. In some embodiments the distal end includes a tip having a taper, nozzle, valve and/or luer lock-like connection for connecting with tubing 584. In some embodiments the distal end includes a pressure fit, friction fit, or threaded connection for connecting with tubing 584. In some embodiments the distal end may be integrally connected or monolithically formed with tubing 584.

Barrel 580 defines a longitudinal axis X1, as shown in FIG. 35. along which the actuator (e.g., plunger 592) moves. In some embodiments, barrel 580 may have cross section configurations, such as, for example, circular, oval, triangular, square, polygonal, irregular, uniform, non-uniform, offset, staggered, undulating, arcuate, variable and/or tapered. In some embodiments, a wall of barrel 580 can be flexible, elastic, semi-rigid or rigid.

In the depicted example barrel 580 includes an interior surface 596. Surface 596 defines a cavity, such as, for example, a passageway 600. In the depicted example, passageway 600 extends along axis X1 between the proximal and distal ends. In one embodiment passageway 600 is configured for disposal of thawed and/or flowable bone growth composition, such as alone or within a container. In some embodiments, passageway 600 may have cross section configurations, such as, for example, circular, oval, triangular, square, polygonal, irregular, uniform, non-uniform, offset, staggered, undulating, arcuate, variable and/or tapered.

In some embodiments, barrel 580 includes visual indicia (e.g., markings), such as, gradations on an outer surface configured to provide a visual indication of the amount of bone growth composition within the application device 500B and/or the amount of bone growth composition that has been injected when in use. In some embodiments, a portion or the entire barrel 580 is transparent to provide a visual indication of the amount of bone growth composition that remains in barrel 580.

Tubing 584, whether a flexible applicator tube or an inflexible hollow needle, is configured for attachment with a tip of barrel 580 adjacent opening 590. As noted herein, the tubing 84 may connect to the tip of barrel 580 using any suitable attachment mechanism, such as, for example, a luer lock connection, a friction fit, a pressure fit, a locking protrusion/recess, locking keyway and/or adhesive. The tip of the tubing (e.g., a flexible tubing or rigid needle) is configured for placement adjacent or proximate to an anatomic site at which bone growth composition is to be applied.

As discussed herein, the tubing 584 defines a cavity or passageway 604. Tubing 584 is attached with the tip of barrel 580 adjacent opening 590 to connect passageway 600 with passageway 604 to form a continuous passageway. Passageway 604 is configured for passage of bone growth composition as discussed herein such that a volume of bone growth composition is expelled via the passageway to the anatomic site of interest. In some embodiments, passageway 604 may have cross section configurations, such as, for example, circular, oval, triangular, square, polygonal, irregular, uniform, non-uniform, offset, staggered, undulating, arcuate, variable and/or tapered. It will be understood that the tubing 584 can be any suitable length depending on the procedure and anatomic target region.

In some embodiments, the tubing 584 may include visual indicia, such as, gradations on an outer surface configured to provide a visual indication of the amount of bone growth composition dispensed. In some embodiments, a portion or the entire tubing 584 is transparent to provide a visual indication of the amount of material that remains in tubing 584.

In some embodiments, the tubing 584 comprises a steering member that allows the tip of the tubing to be manipulated or steered so that an angle of tubing 584 can be changed. In some embodiments, the steerable tip utilizes similar technology as a steerable catheter (e.g., multiple lumens, with one being used for a wire to pull the tip). In some embodiments, an end of tubing 584 can be closed or have an opening and the surface of tubing can have one or a plurality of openings or fenestrations for dispensing bone growth composition. In some embodiments, these openings can be disposed at an angle and/or orientation for dispensing the bone growth composition to the desired anatomic site or sites. In some embodiments, the plurality of fenestrations can also be oriented along the length of the tube so as to give a line of dispensing, rather than a point of dispensing. In some embodiments, the plurality of fenestrations can be approximated by a single long opening.

In some embodiments, the surface of tubing 584 can have one or a plurality of openings for dispensing bone growth composition and the application device 500B can include a handle for orienting the openings. The handle can be rotated, translated or pivoted to the proper orientation for delivery of the bone growth composition.

In some implementations a backfill material may also be employed as part of an application operation to flow through passageway and help expel the selected volume V of bone growth composition into the anatomic site of interest. In particular, such a backfill may be employed to ensure all of the bone growth composition is expelled from the application device 500B and that none of the bone growth composition remains stranded in the syringe barrel 580 and/or tubing 584. In some embodiments, a viscosity of the backfill facilitates driving bone growth composition from passageway 600, through passageway 604 and into a selected anatomic site. In some embodiments, the viscosity of backfill is lower than the viscosity of bone growth composition to facilitate movement of bone growth composition through the passageways. In some embodiments, the viscosity of bone growth composition is equal to the viscosity of the backfill. In some embodiments, the evacuator may include a solid, liquid or gaseous substance, and/or include a mechanical element such as a gasket, disc, stopper or plunger.

In some embodiments, the backfill may comprise, for example, sterile water, glycerol, saline, oil or any polysaccharide; and/or material that is flowable and biocompatible, such as, for example, blood, or blood components including plasma, platelet-rich plasma, buffy coat. In some embodiments, the backfill may comprise, for example, cement to render the application device 500B a single use device and/or disposable. In some embodiments, the backfill may comprise, for example, solid chunks of material that, when injected, flow similar to a liquid.

In one such embodiment, a plunger 592 actuates movement of backfill and bone growth composition within the passageways. The plunger 592 may extend between the proximal and distal ends of the syringe barrel 580. The plunger 592 may include a handle 612 configured for manipulation to translate plunger 592 relative to the wall of barrel 580 within passageway 600. The plunger 592 may also include a plunger seal 616 that slidably engages the wall of barrel 580 such that plunger 592 is movably disposed with opening 588. Plunger seal 616 may be configured to resist and/or prevent backfill and/or bone growth composition from exiting opening 588 of the syringe body.

Plunger seal 616 is configured to translate within passageway 600 and contact backfill and/or bone growth composition. Plunger 592 translates relative to barrel 580 between an initial orientation, as shown in FIG. 35, and a fully or entirely expelled orientation, as shown in FIG. 36. In the initial orientation, plunger 592 is disposed adjacent the backfill and/or bone growth composition. Plunger 592 translates, in the direction shown by arrow A in FIG. 35, such that plunger seal 616 applies a force to the backfill and/or bone growth composition. The force applied by plunger seal 616 causes the bone growth composition to flow from passageway 600 through opening 590. Further translation of plunger 592 causes the bone growth composition into and out of the passageway 604, such as to the anatomic site of interest.

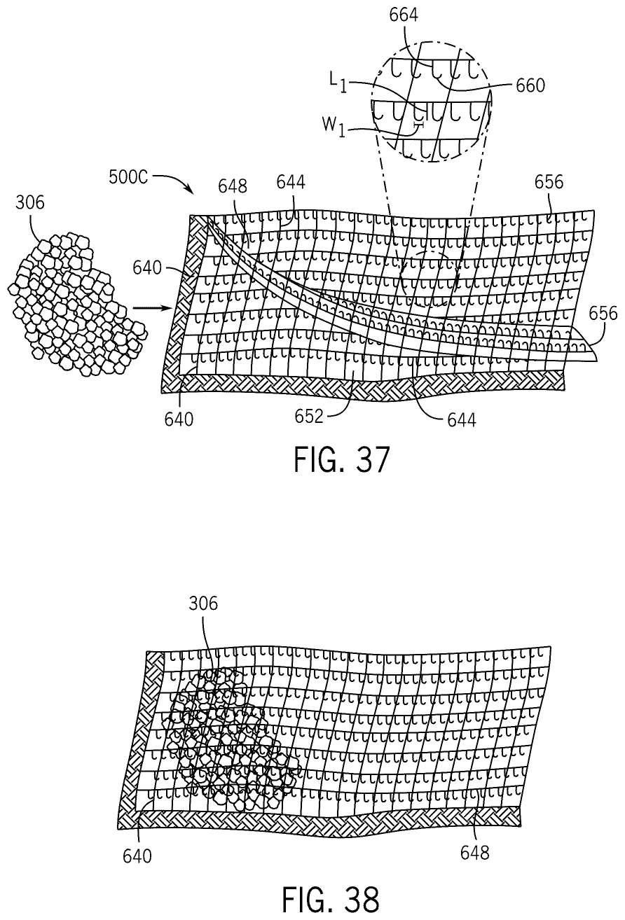

In an alternative embodiment, the application device 500 for the bone growth composition may be provided as a fillable mesh pouch or structure that may be disposed by a clinician at the anatomic site where bone growth is to be stimulated. By way of example, and turning to FIGS. 37 and 38, aspects of one such embodiment are illustrated and described. In this example, FIG. 37 illustrates a perspective view of a partially open configuration of an application device 500C in the form of an implantable structure for enclosing bone growth composition. The implantable structure comprises mesh material defining an inner surface and an outer surface opposing the inner surface. The inner surface is configured to receive a bone growth composition when the inner surface of the mesh is in an open configuration. In one embodiment, a plurality of projections is disposed on or in the inner surface of the mesh. The plurality of projections extends from the inner surface of the mesh and are configured to engage a section of the inner surface of the mesh or a section of the outer surface of the mesh or both sections of the inner and outer surfaces of the mesh in a closed configuration so as to enclose the bone growth composition. With this in mind, FIG. 38 illustrates a perspective view of a closed configuration of the application device 500C of FIG. 37.

With this in mind, and as discussed in greater detail below, in some embodiments, a bone growth composition application device 500C is provided that comprises a mesh material having hook-type polymer features embedded or entangled within the mesh. The hooks provide flexibility in implant diameter and width and can be used to seal and close the application device 500C at varying diameters so that the mesh becomes self-adherent. Any bio-compatible, fully absorbable material can be used to fabricate the mesh, such as, for example, absorbable polymers, such as, poly(lactic acid) (PLA), poly(glycolic acid) (PGA), poly(lactic-co-glycolic acid) (PLGA), polydioxanone (PDO), allogeneic collagen, xenogenic collagen, ceramic or a combination thereof. The mesh can be fabricated using woven or nonwoven techniques, such as needle punching, stitch, thermal or chemical bonding, hydro entanglement, felted or point-bonded, or with additive manufacturing methods (e.g., 3D printing). The mesh has a pore size that is large enough so that cellular transport and formation of new bone is not impeded. However, the pore size is small enough to adequately contain the presently disclosed bone growth composition at an anatomic site of interest. The application device 500C, due to its flexible and configurable construction can be various shapes and can be provided with instruments designed to ease intraoperative use and assembly. Customization of the application device 500C diameter and width enables the application device 500C to be used in a variety of bone fusion repair procedures that use a smaller graft size, such as, minimally invasive midline lumbar fusion, posterior cervical fusion, posterolateral fusion of the transverse process of the human spine and oral maxillofacial repair procedures. In some embodiments, the application device 500C can also accommodate a larger size for longer procedures.

In some embodiments, a mesh is provided that is formed as a bilayered envelope with two of the four sides closed during fabrication. The closed sides can be joined by, for example, ultrasonic welding, heat sealing or suturing. The two of the four sides being closed simplifies assembly by providing a preformed pocket configured for placement of bone growth composition, as discussed herein.

In some embodiments, a device may be employed to hold the mesh while filling the mesh with bone growth composition. The device can be a tray and can be positioned in an upright configuration during placement of the bone growth composition into the mesh. The tray can be a thermoform tray comprising a central trough. The tray can also be made of metal or a polymer. The tray may or may not be a part of a sterile packaging for the application device 500C. The tray may also comprise projections or other features to clip and/or hold the application device 500C in a desired spatial arrangement.

With the preceding in mind, and turning to FIGS. 37 and 38, an application device 500C is provided that is customizable, self-sealing, self-adherent and is configured for sealing and enclosing a bone growth composition 306, as described herein. The application device 500C is configured to be cut and shaped in any size and diameter for a particular target anatomic site. The bone growth composition 306 described herein, can be fully or partially enclosed by the mesh 640.

The application device 500C comprises a mesh 640 having an inner surface 644 and an outer surface 648 opposing the inner surface 644. The inner surface 644 is configured to receive bone growth composition 306 when the inner surface 644 of the mesh 640 is in an open configuration, as shown in FIG. 37. Mesh 640 comprises, consists essentially of, or consists of material 652 that may be a non-woven material or, alternatively, a material formed of woven or knitted monofilament or multifilament threads. That is, as used herein, a woven or knitted structure is made of filaments, such as interleaved or entangled filaments. As discussed herein, the mesh 640 may be such a woven or knitted structure.

In certain embodiments the mesh 640 can be made of a material 652 or fabric that, in some aspects can be shape memory, felted, point-bonded, additive manufactured, such as 3-D printed or a combination thereof. In some aspects, the mesh 640 comprises, consists essentially of, or consists of nonwoven material, which is electrospun, made by needling material to create a felt, point-bonded, spun-bonded or a combination thereof. In various embodiments, the mesh 640 comprises, consists essentially of or consists of a nonwoven material or fabric and in some aspects, can be in a sheet form. In various aspects, a nonwoven material is a manufactured sheet, web or batt of directionally or randomly orientated fibers, bonded by friction, and/or cohesion and/or adhesion. The fibers may be of natural or man-made origin.

In nonwoven embodiments, the material 652 is directly made from fibers or filaments without the need of converting the fibers or filaments into yarns. Nonwoven materials or fabrics broadly relate to sheet or web structures bonded together by entangling fiber or filaments (and by perforating films) mechanically, thermally, or chemically. The term “nonwoven” generally refers to a flat textile structure made up of individual fibers that, in contrast to woven, knit and knitted fabrics, is not made up of filaments. A nonwoven material can be made directly from a web of fiber, without the yarn preparation necessary for weaving and knitting. In a nonwoven material, the assembly of textile fibers is held together by mechanical interlocking in a random web or mat; by fusing of the fibers, as in the case of thermoplastic fibers; or by bonding with a cementing medium such as starch, casein, rubber latex, a cellulose derivative or synthetic resin. Initially, the fibers may be oriented in one direction or may be deposited in a random manner. This web or sheet is then bonded together mechanically, thermally, or chemically. Fiber lengths can range from 0.25 inch to 6 inches for crimped fibers up to continuous filament in spunbonded fabrics.

In some embodiments, the material 652 used to make the mesh 640 described herein can be subjected to finishing operations such as coating and laminating, calendaring, and embossing to impart particular surface properties, corona and plasma treatments to change the wetting properties of the fabric, wet chemical treatments to impart anti-static properties, anti-microbial properties, flame retardant properties.

The mesh 640 can be absorbable and can be made from a material, including, but not limited to at least one of poly(lactic acid) (PLA), poly(glycolic acid) (PGA), poly(lactic-co-glycolic acid) (PLGA), polydioxanone (PDO), allogeneic collagen, xenogenic collagen, ceramic or a combination thereof. The mesh 640 when formed from an absorbable material may be substantially resorbed within 2 weeks, within 4 weeks, within 12 weeks, within 16 weeks, within 20 weeks, within 24 weeks, within 28 weeks, within 32 weeks, within 36 weeks, within 40 weeks, within 44 weeks, within 48 weeks, within 52 weeks or within any other suitable time frame.

The material and configuration of the mesh 640 may be selected or adjusted based on desired release characteristics. Specific properties of the mesh 640 that may be adjusted include thickness, permeability, porosity, strength, flexibility, and/or elasticity. In some embodiments, the thickness and porosity of the mesh 640 may contribute to its strength, flexibility, and elasticity. In some embodiments, the mesh 640 may be made of, incorporate, or include a squishy, moldable, sticky, and/or tacky material to facilitate placement and packing of the bone growth composition to an anatomic site of interest.

The average molecular weight of the polymer used to make the mesh 640 can be from about 1,000 to about 10,000,000; or about 1,000 to about 1,000,000; or about 5,000 to about 500,000; or about 10,000 to about 100,000; or about 20,000 to 50,000 g/mol. In some embodiments, the molecular weight of the polymer is 1,000, 2,000, 3,000, 4,000, 5,000, 6,000, 7,000, 8,000, 9,000, 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, 100,000, 125,000, 150,000, 175,000, 200,000, 225,000, 250,000, 275,000, 300,000, 325,000, 350,000, 375,000, 400,000, 425,000, 450,000, 475,000, 500,000, 525,000, 550,000, 575,000, 600,000, 625,000, 650,000, 675,000, 700,000, 725,000, 750,000, 775,000, 800,000, 825,000, 850,000, 875,000, 900,000, 925,000, 950,000, 975,000 and/or 1,000,000 Daltons.

The mesh 640 may have varying degrees of permeability. It may be permeable, semi-permeable, or non-permeable. Permeability may be with respect to cells, to liquids, to proteins, to growth factors, to bone morphogenetic proteins, or other substances. The mesh 640 may be 1 to about 30% permeable, from about 30 to about 70% permeable, or from about 70 to about 95% permeable. The mesh 640 may be 1, 5, 10, 15, 20, 25, 30, 35, 40, 45, 50, 55, 60, 65, 70, 75, 80, 85, 90, 95, 96, 97, 98, or 99% permeable. In alternative embodiments, the mesh 640 may comprise a substantially solid structure, such as a polymer structure with a chamber, or a spun cocoon.

The mesh 640 can be a porous mesh such that fluid transfer and cell infiltration can occur so that osteoblasts can manufacture bone graft. The porous mesh can have a pore size of from about 1 micron to about 2000 microns, from about 1 micron to about 1500 microns, from about 1 micron to about 1000 microns, from about 1 micron to about 500 microns, from about 1 micron to about 250 microns, from about 100 micron to about 2000 microns, from about 150 to about 1500 microns, from about 200 to about 1000 microns, from about 250 to about 500 microns. In some embodiments, the pore size can be about 1, 10, 20, 50, 80, 100, 120, 150, 180, 200, 220, 250, 280, 300, 350, 400, 450, 500, 550, 600, 650, 700, 750, 800, 850, 900, 950, 1000, 1250, 1450, 1650, 1850, and/or 2000 microns.

The application device 500C as discussed herein may include projections (e.g., hooks, grips, etc.). The projections, when present, allow the application device 500C having the mesh 640 and bone growth composition 306 therein to be rolled or folded into a closed configuration (e.g., tubular configuration) and retained in the closed configuration for implanting. In this manner, the application device 500C can be customized in terms of shape and/or size to match the conformation of the target region for deployment of the application device 500C, e.g., a bone defect or void in which bone growth is desired.

A plurality of projections, such as, for example, hooks 656 are disposed in at least a portion of the inner surface 644 of the mesh 640, the outer surface 648 of the mesh 640 or both the inner surface and outer surface of the mesh 640, the plurality of projections 656 extending from at least the portion of the inner surface 644, the outer surface 648 of the mesh 640 or both the inner surface and outer surface of the mesh 640. Hooks 656 extend from at least a portion of the inner surface 644 of the mesh. The hooks 656 are configured to engage a section of the inner surface 644 of the mesh 640 or a section of the outer surface 648 of the mesh 640 or both sections of the inner and outer surfaces of the mesh 640 when the mesh 640 is in a closed configuration so as to enclose the bone growth composition 306.

Each of the hooks 656 include a proximal end 660 and a distal end 664, as shown in FIG. 37. The distal end 664 is fixed on or in at least a portion of the inner surface 644 of the mesh 640 and the proximal end 660 engages one or a plurality of projections that are disposed on or in at least a portion of an opposing inner surface of the mesh 640. The plurality of projections or hooks 656 can be made from the same material as the mesh 640 or a different material than the mesh 640 and can be entangled in the mesh. In various embodiments, when entangled, the plurality of projections, hooks or grips are intertwined or inextricably connected to the mesh 640 so that they can catch each other or other portions of the inner and/or outer surface of the mesh 640 and the mesh becomes self-adherent.

In various embodiments, the hooks 656 comprise, consist essentially of, or consist of monofilament material that is more rigid than the mesh 640 and the mesh can be in a sheet form. In various embodiments, the plurality of projections can be prepared from poly(glycolic acid) (PGA), poly(lactic acid-glycolic acid) (PLGA), polyurethane, polylactide (PLA) (also known as polylactic acid, lactic acid polymer), Velcro® or other resorbable copolymers. The hooks 656 can be added to the entire inner surface 644 of the mesh 640 or a portion of the inner, outer or both surfaces of the mesh 640 after the mesh 640 is manufactured via a coating, 3D printing and/or screen printing.

In some instances, the mesh 640 can be created initially, and the plurality of projections can be subsequently added to the inner surface 644, outer surface 648 or both the inner and outer surface of the mesh 640 and configured to engage a section of either or both surfaces in a closed configuration so as to enclose bone growth composition 306. In other instances, the plurality of projections is initially prepared from monofilament material and then subsequently added to the material of the mesh 640 in another step.

The plurality of projections can also be grips, teeth, spikes, barbs, protuberances, prongs, clips, spurs, quills, pins, or a combination thereof. These projections can contact the opposing surface of the mesh 640 and seal the mesh 640 on contact with the opposing surface of the mesh 640. These projections are configured to hold sections of the inner surface 644, outer surface 648, or both inner and outer surface of the mesh 640. In some embodiments, the density of the plurality of projections can be from about 1 to about 500 projections per inch, from about 1 to about 400 projections per inch, from about 1 to about 350 projections per inch, from about 1 to about 300 projections per inch, from about 1 to about 250 projections per inch, from about 1 to about 200 projections per inch, from about 1 to about 150 projections per inch, from about 1 to about 100 projections per inch, or from about 1 to about 50 projections per inch. In some embodiments, the density of the plurality of projections can be from about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 to about 500 projections per inch. The density of the plurality of projections may be uniform throughout the inner surface 644 of the mesh 640 or may not be uniform.

As illustrated in the detail of FIG. 37, each of the plurality of projections may have a stalk portion of selected length Li of, in some embodiments, from about 0.001 millimeters (mm) to about 10 mm, from about 0.005 mm to about 3 mm, or from about 0.01 mm to about 2 mm. Each of the plurality of projections may have a selected length of from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4.0, 4.5, 5 mm to about 10 mm. Each of the plurality of projections may have the same or different lengths.

Each of the plurality of projections may have a selected hook portion having a width W1 of, in some embodiments, from about 0.001 to about 2 mm, from about 0.003 mm to 0.05 mm, or from about 0.005 mm to about 0.08 including or excluding a proximal end comprising a hook portion. Each of the plurality of projections may have, in some embodiments, a selected width of from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.5, mm to about 2 mm including or excluding a proximal end comprising a hook portion. Each of the plurality of projections may have the same or different widths.

The projections are configured for mating engagement with opposing projections or other parts of the mesh 640. For example, a plurality of projections fixed on or in at least a portion of the inner surface 644 of the mesh 640, slidably engage via their proximal end 660 with proximal ends 660 of a plurality of projections or gaps between a plurality of projections that are disposed on or in at least a portion of an opposing inner surface of the mesh 640 (FIG. 37), or a section of the mesh 640. In some embodiments, the opposing plurality of projections slide and matingly engage in 2 projections to 1 projection engagement, in 3 projections to 1 projection engagement, in 4 projections to 2 projections engagement and/or 1 projection to 1 projection engagement.

The plurality of projections is flexible so as to facilitate mating engagement, as described above. The flexibility of the plurality of projections are measured by tensile elasticity or its modulus of elasticity. The plurality of projections, in some embodiments, have a modulus of elasticity of about 1×10² to about 6×10⁵ dyn/cm², or 2×10⁴ to about 5×10⁵ dyn/cm², or 5×10⁴ to about 5×10⁵ dyn/cm².

The mating engagement of the projections to opposing projections or the inner or outer surface of the mesh 640 may have an attachment strength such as a sheer strength, lateral sheer and/or peel strength of about 20 to about 400 Newtons (N), about 50 to about 350 N, about 50 to about 300 N, about 75 to about 300 N, about 75 to about 200 N, or about 100 to about 150 N. The mating engagement of the projections to opposing projections or the inner or outer surface of the mesh 640 may have an attachment strength such as a sheer strength, lateral sheer and/or peel strength of from about 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, to about 300 N.

In FIGS. 37 and 38, FIG. 37 illustrates an open or partially open configuration of mesh 640 and the bone growth composition 306 is being filled into the mesh 640. FIG. 38 illustrates a closed configuration to the mesh 640 and the bone growth composition 306 is enclosed by the mesh 640.

In some embodiments, as shown in FIG. 37, the plurality of projections is disposed on or in the entire inner surface 644 of the mesh 640. In some embodiments, the plurality of projections is disposed in or on discrete regions of the inner and/or outer surface of the mesh 640. In other embodiments, the plurality of projections is entangled within the inner surface 644, outer surface 648 or both the inner surface and outer surface of the mesh 640. In some aspects, the plurality of projections protrudes from the inner, outer or both surfaces and can engage other projections or material. In yet other aspects, the plurality of projections is part of a connected monofilament structure, wherein the monofilament structure is attached to the mesh 640 or is woven or knit. The mesh 640 is sealed by hand or machine by bringing opposing projections together at the discrete regions and applying pressure to those regions to seal the mesh 640 and enclose or partially enclose the bone growth composition inside the mesh 640 as shown by the sequence of FIGS. 37 and 38.

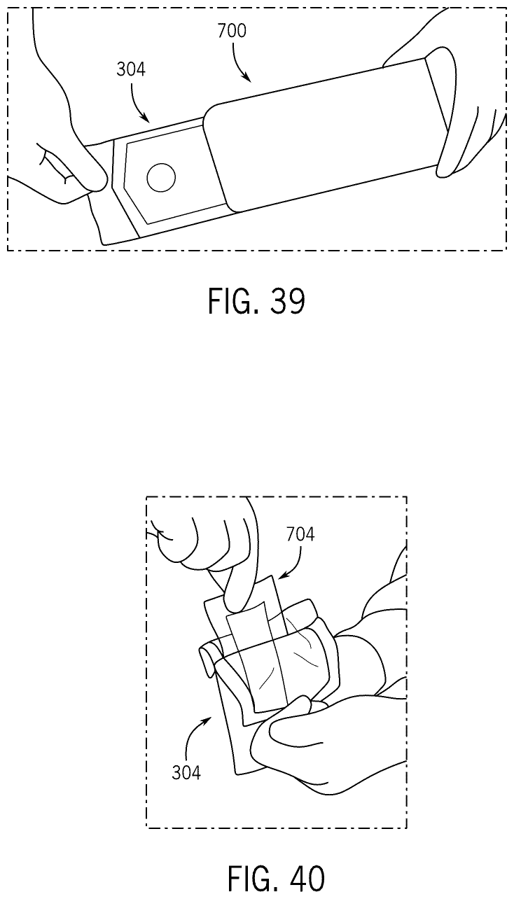

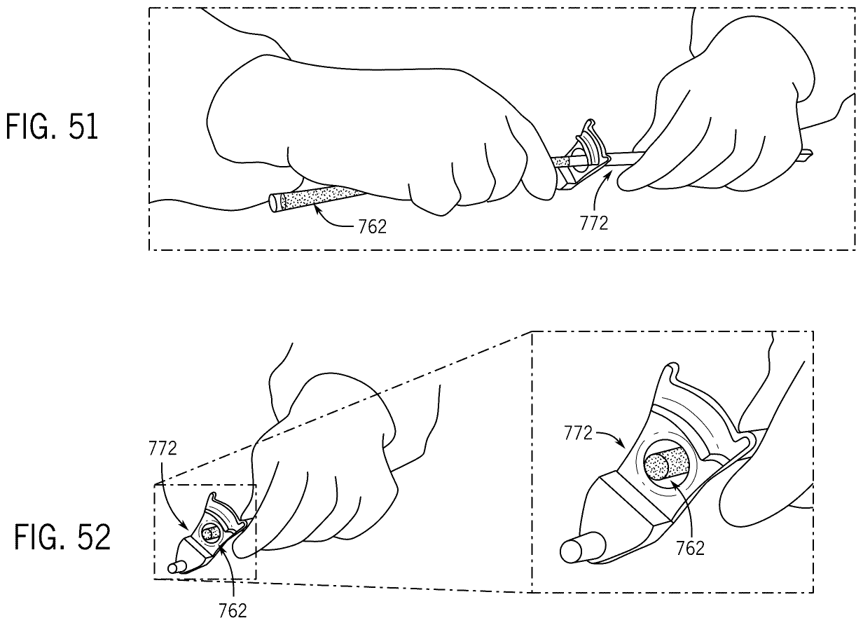

With the preceding examples of application devices 500 in mind, an example walk-through of a bone growth composition application procedure is illustrated in FIGS. 39-56. The depicted walk-through visually illustrates aspects of the packaging, unpackaging, preparation, and application bone growth composition, as discussed herein, in accordance with one or more embodiments. By way of example, and turning to FIG. 39, in one embodiment bone growth composition (e.g., 1 cc, 2.5 cc, 3 cc, 5 cc, 10 cc, 15 cc, and so forth) may be provided in a sealed pouch 304 that in turn may be provided or shipped in a box 700 or other packaging. In practice box 700 may be sealed (e.g., shrink-wrap sealed) to protect against moisture or other environmental conditions.

In the depicted example, other devices discussed herein (e.g., cannulas) are not shown as being provided in the same packaging as the bone growth composition. However in other embodiments other tools, such as cannulas, may be included in the same packaging (e.g., box or carton) as the bone growth composition, (such as in a separate sealed pouch within a box). In practice, the different sizes of packaging may include different materials. By way of example, a 1 cc box 700 as depicted in FIG. 39 may include a pouch 304 of bone growth composition while larger sizes (e.g., 2.5 cc, 3 cc, 5 cc, and so forth) may include not only the designated quantity of bone growth composition (e.g., a pouch of bone growth composition), but also devices or tools such as cannulas for application of the bone growth composition. Examples of packaging for such tools and devices is described elsewhere herein. In such an example, as shown in FIG. 39, the user opens the box 700 to remove the pouch 304 prior to use. In practice, the box 700 may be stored in a freezer or otherwise maintained at the specified temperature range (e.g., −70 to −80° C.) prior to opening.

In certain embodiments, and as illustrated in FIG. 40, the bone growth composition may be “double-pouched” (or “triple-pouched” as described elsewhere herein), with the pouch 304 containing a sterile sealed inner pouch 704 in which the bone growth composition 306 is contained. In one embodiment the inner pouch 704 may be a nylon pouch, but in practice the inner pouch 704 may be fabricated using any medically suitable polymer or composition. In one such example, using suitable sterile techniques, the pouch 304 may be opened (e.g., peeled open using unsealed flaps at an end, cut open at a visually marked indicator, torn open along a perforation, and so forth) and the sterile inner pouch 704 containing the bone growth composition 306 may be aseptically accessed and transferred to a sterile field.

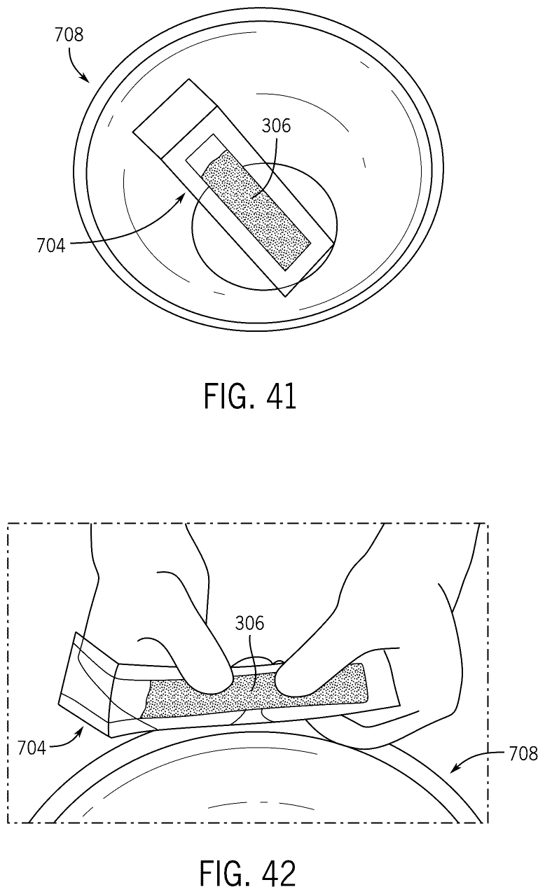

In one such example and turning to FIG. 41, within the sterile field warm, sterile isotonic solution may be poured (or may be pre-poured) into a sterile basin 708 (e.g., a 1 liter sterile basin) or other suitable container. The starting temperature of the solution may, in one implementation, be 37° C.±2° C. The inner pouch 704 containing the frozen bone growth composition 306 is submerged in the sterile isotonic solution within the sterile basin. The bone growth composition 306 is thawed until the bone growth composition is flowable (e.g., flows freely) or is otherwise sufficiently thawed. Depending on the amount of bone growth composition 306, the thawing process may be 3 to 6 minutes or longer (e.g., 10 to 15 minutes). In practice, the temperature of the isotonic solution does not have to be maintained during the thawing process. As shown in FIG. 42, the inner pouch 704 may be manually manipulated (e.g., massaged) to determine that the bone growth composition 306 has sufficiently thawed for application (e.g., is pliable or flowable). Additional warming, if needed, may be provided by returning the inner pouch 704 to the basin 708 or by holding the inner pouch 704 in sterile gloved hands until sufficiently thawed. Upon thawing, the bone growth composition should be malleable and flow freely. In accordance with presently contemplated and disclosed embodiments, the bone growth composition does not require the removal of cryoprotectant prior to application (e.g., implantation).

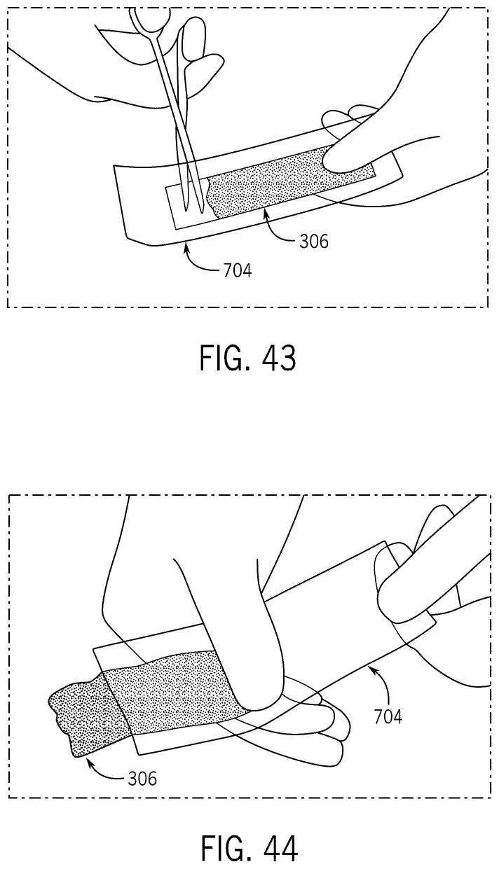

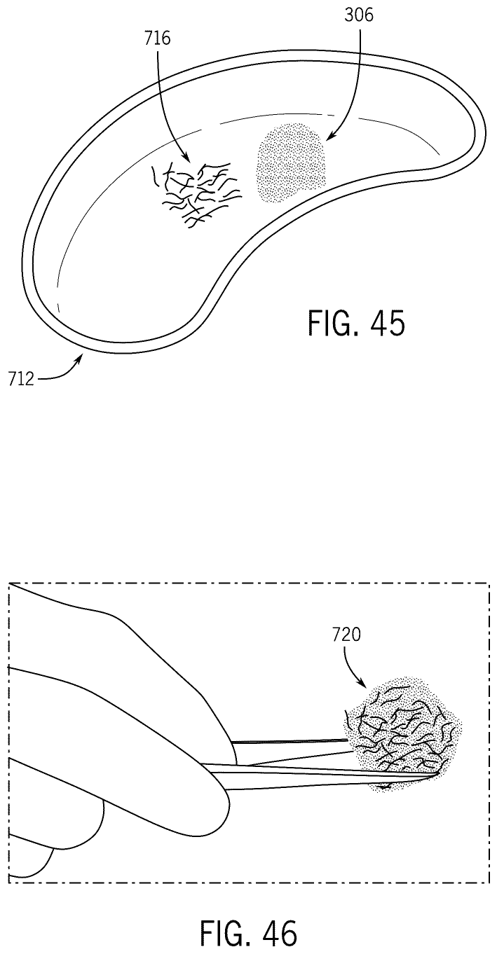

Once the bone growth composition 306 is sufficiently thawed, the inner pouch 704 is removed from the basin 708 and opened (e.g., peeled open using unsealed flaps at an end, cut open at a visually marked indicator, torn open along a perforation, and so forth). An example of this process is illustrated in FIG. 43 where the inner pouch 704 is cut using a suitable sterile implement (e.g., sterile scissors). Once the inner pouch 704 is opened, the bone growth composition 306 may be extracted into a separate surgical basin 712 (FIG. 45). By way of example, the thawed bone growth composition 306 may be squeezed out of the inner pouch 704, as shown in FIG. 44) or may be scooped out of the inner pouch 704 with a sterile instrument. In certain embodiments, the bone growth composition 306 may be adjusted by addition of isotonic solutions or removal of excess fluid.

In certain embodiments, the bone growth composition 306 may be mixed or otherwise combined with local autograft 716 and/or bone marrow aspirate (BMA) prior to application (e.g., implantation). An example of such a combined 720 bone growth composition and local autograft is illustrated in FIG. 46. In other embodiments, the bone growth composition is applied without being combined with autograft or BMA.

With respect to the application device or devices, such devices may be provided with the bone growth composition, such as part of a kit, or separately. By way of further example, a visual walk-through of steps for using the thawed bone growth composition 306 with an application device, such as the application device 500A discussed with respect to FIG. 34, is provided herein. Such an application device 500A may be suitable for use with bone growth composition 306 provided in suitable pre-packaged amounts, such as 2.5 cc, 5 cc, 10 cc, 15 cc, and so forth.

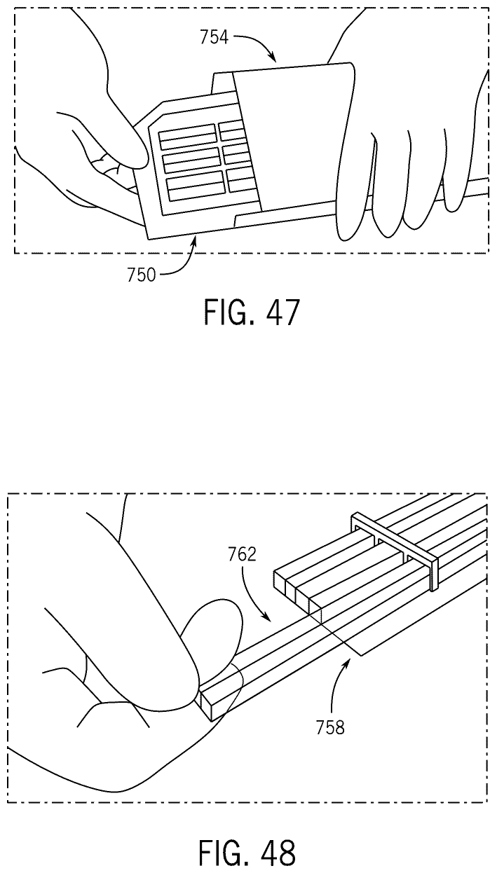

As discussed herein, in certain implementations the bone growth composition 306 may be mixed with local autograft and/or BMA prior to being loaded into an application device, such as into single-use cannulas provided for loading into an application device 500. By way of example, in certain such embodiments the autograft may be milled to below a threshold size (e.g., to less than 3.2 mm) and the combined product may be less than 50% autograft by volume so as to reduce the likelihood of clogging the cannulas. With this in mind, a walk-through of certain steps in a bone-growth composition application process are provided to illustrate an example workflow. In one such implementation, a cannula tray 750 may be removed from a package or box in which it is shipped or stored. In practice, there may be an outer package or box that serves to identify the contents and to protect an inner box 754 in which a cannula tray 750 is provided. In certain such examples, bone growth composition, packaged as described above, may also be provided in a separate box or pouch provided within the outer package or box. Shrink wrap or other sealing material may be removed from the box 754 as part of this process of removing the cannula tray 750. Aspects of this process are illustrated in FIG. 47 where a cannula tray 750 is illustrated as it is removed from box 754.

In the depicted example, the cannula tray 750 is depicted as being sealed and containing within the sterile sealed environment a die card 758 in which paired, joined cannulas 762 are contained. In this example, using proper sterile technique the cannula tray 750 may be unsealed and the die card 758 removed and transferred to the sterile field. Once in the sterile field, the cannulas 762 may be removed from the die card 758 and separated for use.

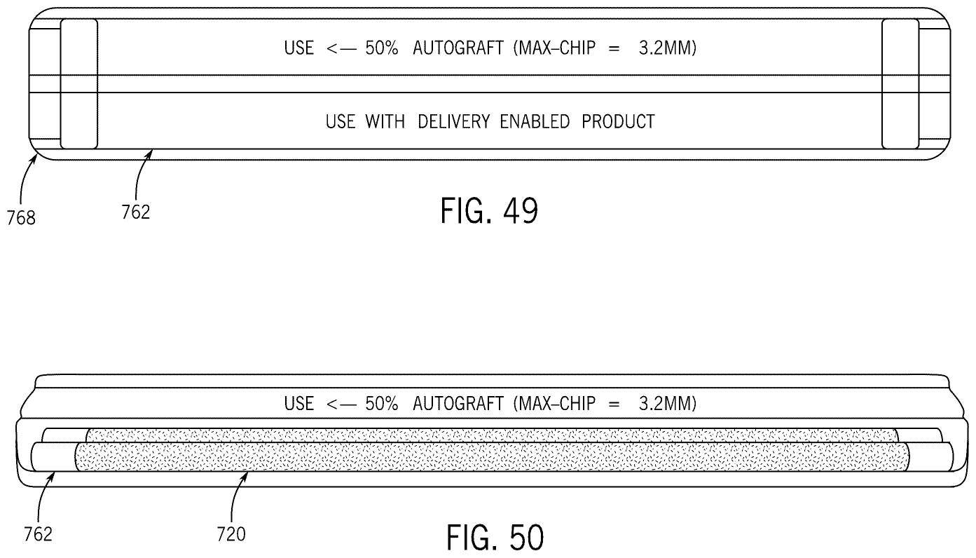

Turning to FIGS. 49 and 50, an example of a cannula loading procedure is depicted. The empty cannulas 762 are illustrated in a paired configuration in FIG. 49. Before loading, the cannulas 762 may be opened and closed once or more than once to make closing of the cannula 762 easier once loaded with the bone growth composition 306 or bone growth composition combined with autograft and/or BMA (combination 720). To facilitate loading of the cannulas 762, the cannulas 762 may be placed into fitted grooves or receptacles (e.g., concave receptacles) of a loading platform 768, which may be sized to accommodate one, two, or more cannulas and which may be associated with the respective application device 500. The flowable bone growth composition 306 or bone growth composition combined with autograft and/or BMA may be inserted into a cannula 762 and may be evenly or uniformly spread within the cannula 762. The flowable bone growth composition 306 or bone growth composition combined with autograft and/or BMA may be distributed so as to stay within the top of the cannula curvature. Once a cannula 762 is filled, it may be closed, with excess material being removed as needed to achieve closure of the cannula 762.

In the depicted example, an application device 500A is shown by way of example. In this example, the application device 500A includes a funnel structure 772 into which a cannula 762 may be loaded and which incorporates the delivery tube 504 of the application device 500A (FIG. 34). In this example, and as illustrated, a loaded cannula 762 may be inserted into a corresponding receptacle of the funnel structure 772. In some implementations, and as shown in FIG. 52, the loaded cannula 762 will protrude or extent outside of the loading opening of the funnel structure 772, such as protruding 3 mm, 4 mm, 5 mm, or 6 mm. An internal ledge or other protrusion internal to the funnel structure 772 on the opposing end may be provided to prevent the loaded cannula 762 from extending out of the opposing end of the delivery tube 504.

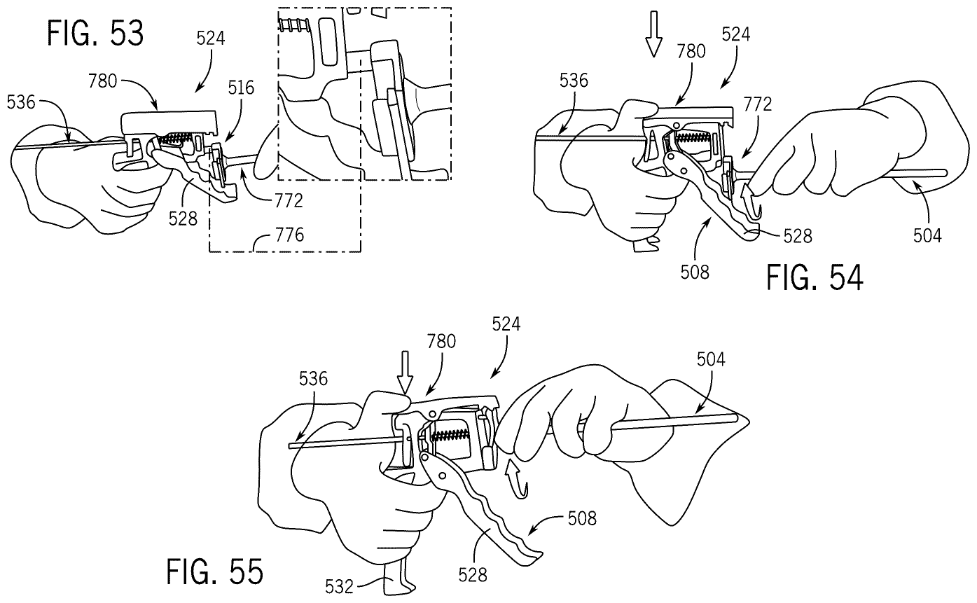

The funnel structure 772 incorporating the delivery tube 504 may be attached to the application device 500A, such as to frame 524, once loaded. In the depicted example of FIG. 53, a funnel pin 776 may be slid into a body socket of the receiving mechanism 516 at a perpendicular angle. Turning to FIGS. 54 and 55, while depressing a clasp 780 on the frame 524, the funnel structure 772 is rotated into alignment with the frame 524. Once aligned, the clasp 780 is released to secure the funnel structure 772 to the frame 524. Once secured in this manner, the plunger (e.g., rod 536) may be advanced into and through the loaded cannula 762 by manipulating (e.g., squeezing) the handles 528, 532 of the actuation mechanism 508. In this manner, bone growth composition 306 or the combination 720 of bone growth composition with autograft and/or BMA may be expelled from the cannula 762 and through the open distal end 512 of delivery tube 504 of the application device 500A.

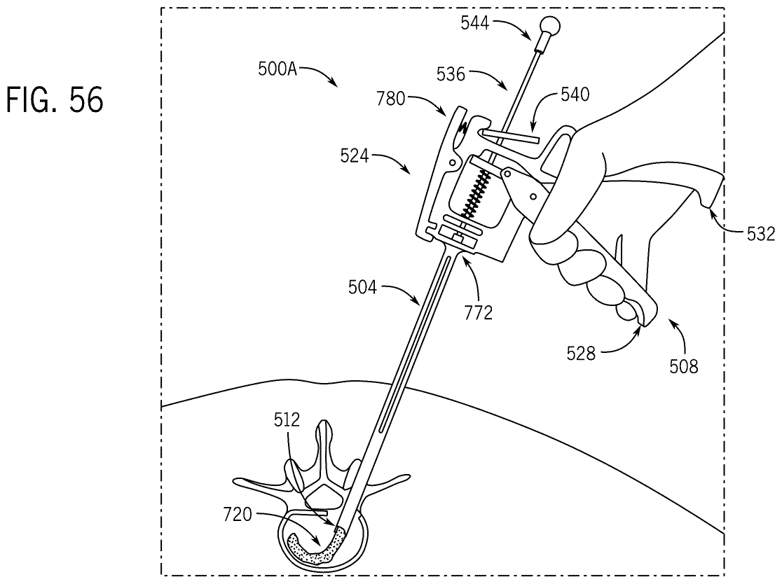

Once the application device 500A is loaded in this manner, the plunger (e.g., rod 536) may be advanced using the actuation mechanism 508 during a medical procedure, as illustrated in FIG. 56. In this example, the combination 720 of bone growth composition with autograft and/or BMA is expelled through the open distal end 512 of delivery tube 504 into a void or region of the subject and may be delivered by operation of the actuation mechanism 508 until the void is filled, until the cannula(s) 762 are empty, or until the target amount of bone growth composition is otherwise delivered.

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.”

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims. Further, it should be understood that certain elements of the disclosed embodiments may be combined or exchanged with one another.

What is claimed is:

1. A package, comprising:
an inner pouch comprising a bone growth composition within a sealed and sterile environment, the bone growth composition comprising:
demineralized bone fibers generated from a donor bone tissue;
cancellous chips and/or corticocancellous chips generated from the donor bone tissue; and
a cryosolution comprising glycerol in Lactated Ringer's solution, wherein a glycerol content of the cryosolution is equal to or less than 25% by volume.

2. The package of claim 1, further comprising a pouch within which the inner pouch is sealed or placed.

3. The package of claim 2, further comprising an outer pouch within which the pouch is sealed or placed.

4. The package of claim 2, wherein the pouch is configured to be opened by one of having two layers forming the pouch being peeled apart, being cut open, or being torn open along a perforation provided on the pouch.

5. The package of claim 2, wherein the pouch further comprises a rubberized seal configured to allow insertion of a temperature modulating medium internal to the pouch but external to the inner pouch.

6. The package of claim 1, wherein the inner pouch comprises one or more drainage features that, when exposed, facilitate drainage of the cryosolution from the inner pouch.

7. The package of claim 6, wherein the inner pouch and pouch are formed integral with one another.

8. The package of claim 6, wherein the inner pouch further comprises a resealing feature configured to allow resealing of the inner pouch after opening.

9. The package of claim 1, wherein the inner pouch is configured to be opened by one of having two layers forming the inner pouch being peeled apart, being cut open, or being torn open along a perforation provided on the inner pouch.

10. The package of claim 1, wherein the inner pouch contains 1 cc, 2.5 cc, 5 cc, 10, cc, or 15 cc of bone growth composition.

11. The package of claim 1, further comprising:
a sealed cannula tray; and
a die card provided within the cannula tray, wherein a plurality of cannulas is held within the die card and wherein each cannula is configured to receive the bone growth composition or a combination of the bone growth composition and one or both of local autograft or bone marrow aspirate.

12. A method of loading a cannula, comprising:
preparing an amount of bone growth composition for loading into an application device; and
inserting the amount of bone growth composition or a combination of the bone growth composition and one or both of local autograft or bone marrow aspirate into the cannula, wherein the bone growth composition comprises: demineralized bone fibers generated from a donor bone tissue; cancellous chips and/or corticocancellous chips generated from the donor bone tissue; and a cryosolution comprising glycerol in Lactated Ringer's solution, wherein a glycerol content of the cryosolution is equal to or less than 25% by volume.

13. The method of claim 12, wherein preparing the amount of bone growth composition comprises mixing the bone growth composition and one or both of local autograft or bone marrow aspirate prior to insertion into the cannula.

14. The method of claim 12, further comprising, prior to inserting the amount of bone growth composition or the combination of bone growth composition and one or both of local autograft or bone marrow aspirate into the cannula, positioning the cannula on a loading tray, wherein the loading tray comprises at least one groove configured to stabilize the cannula when positioned on the loading tray.

15. The method of claim 12, further comprising closing the cannula once filled with the bone growth composition or the combination of bone growth composition and one or both of local autograft or bone marrow.

16. The method of claim 12, further comprising:
after insertion of the bone growth composition or the combination of bone growth composition and one or both of local autograft or bone marrow, inserting the cannula into a delivery tube of an application device;
securing the delivery tube containing the cannula to the application device; and
operating the application device to dispense bone growth composition or the combination of bone growth composition and one or both of local autograft or bone marrow from the cannula to an anatomic site of a patient.

17. The method of claim 12, wherein inserting the amount of bone growth composition or the combination of the bone growth composition and one or both of local autograft or bone marrow aspirate into the cannula comprises autoloading the cannula with a measured amount of bone growth composition and one or both of local autograft or bone marrow aspirate according to volumetric measurement marks provided on the cannula.

18. The method of claim 12, wherein the application device is a syringe or a bone-grafting injection gun.

19. The method of claim 16, wherein the anatomic site comprises a spine region.

20. The method of claim 16, wherein the anatomic site comprises an area of the patient anatomy comprising a bone defect to be repaired.

21. A mesh pouch, comprising:
two layers of a mesh material secured along one or more edges so as to define an interior space;
a plurality of projections configured to secure the two layers of mesh material along unsecured edges and allowing mesh pouch to be transitioned between an open and a closed configuration; and
a volume of bone growth composition or a combination of the bone growth composition and one or both of local autograft or bone marrow aspirate positioned between the two layers and secured within the interior space, wherein the bone growth composition comprises: demineralized bone fibers generated from a donor bone tissue; cancellous and/or corticocancellous chips generated from the donor bone tissue; and a cryosolution comprising glycerol in Lactated Ringer's solution, wherein a glycerol content of the cryosolution is equal to or less than 25% by volume.

22. The mesh pouch of claim 21, wherein the two layers of the mesh material are secured using the plurality of projections so that one or both of a size or a shape of the mesh pouch conforms to an opening at an anatomic region.

23. The mesh pouch of claim 21, wherein the mesh material comprises a woven, nonwoven, or knitted mesh material.

24. A pouch comprising a bone growth composition within a sealed and sterile environment, the pouch comprising:
one or more drainage features that, when exposed, facilitate drainage of a cryosolution from the pouch;
wherein the bone growth composition comprises:
demineralized bone fibers generated from a donor bone tissue;
cancellous and/or corticocancellous chips generated from the donor bone tissue; and
the cryosolution, wherein the cryosolution comprises glycerol in Lactated Ringer's solution, wherein a glycerol content of the cryosolution is equal to or less than 25% by volume.

25. The pouch of claim 24, wherein the pouch is formed integrally with an additional pouch such that the pouch and the additional pouch are in fluid communication when the drainage features are exposed.

26. The pouch of claim 24, further comprising:
a resealing feature configured to allow resealing of the pouch after opening.
